(19) United States
(12) Patent Application Publication
(10) Pub. No.: US 2024/0082129 A1
(43) Pub. Date: Mar. 14, 2024

(54) HAIR COLORING COMPOSITIONS SUITABLE FOR FACIAL HAIR

(71) Applicant: L'OREAL, Paris (FR)

(72) Inventors: Taylor Katherine HART, New York, NY (US); Kimberly DREHER, Brielle, NJ (US); Brian Michael KLUCK, Piscataway, NJ (US); Minli SHI, Jersey City, NJ (US)

(21) Appl. No.: 17/823,603

(22) Filed: Aug. 31, 2022

(51) Int. Cl. A61K 8/41; A61K 8/34; A61K 8/49; A61Q 5/10

(52) U.S. Cl. CPC A61K 8/411; A61K 8/342; A61K 8/4953; A61Q 5/10; A61K 2800/30; A61K 2800/882

(57) ABSTRACT

The present disclosure relates to hair coloring base compositions and ready-to-use hair coloring compositions comprising 2-methoxymethyl-P-phenylenediamine, an oxidative dye precursor. The hair coloring base composition is mixed with a developer composition to form a ready-to-use hair coloring composition having a lower than typical pH, 7. These compositions are effective for coloring hair, especially beard hair. The color imparted to the hair is long-lasting (tenacious) and skin sensitivity due to the coloring process is minimized or prevented. Methods for making the composition and methods for coloring hair, especially beard hair, are also disclosed.

### FIELD OF THE DISCLOSURE

The present disclosure relates to hair coloring base compositions and ready-to-use hair coloring compositions comprising 2-methoxymethyl-P-phenylenediamine, an oxidative dye precursor. Methods for making the compositions and methods for coloring hair, especially beard and moustache hair, are also disclosed.

### SUMMARY

There are various types of hair coloring compositions for temporarily, semi-permanently, and permanently coloring hair. Most types of hair coloring composition for permanently coloring hair are oxidative hair coloring compositions that use two parts. The first part is a hair coloring base composition that contains oxidative dye precursors (sometimes called “oxidative dyes”) and couplers, which interact with the oxidative dye precursors. The second part is a developer composition containing an oxidizing agent like hydrogen peroxide. The two parts are mixed immediately prior to use forming a ready-to-use hair coloring composition to activate the composition for coloring the hair. Thus, ready-to-use hair coloring compositions can be called “active hair coloring compositions.” The oxidative dye precursors and oxidizing agents from the active hair coloring compositions diffuse into the hair shaft, where color formation takes place through a cascade of chemical reactions. The oxidative dye precursors are oxidized by the oxidizing agents and form reactive intermediates. Couplers, which are relatively stable to oxidizing agents, react with the intermediates resulting in vibrant coloring molecules.

The hair coloring compositions of the instant case include 2-methoxymethyl-P-phenylenediamine, an important oxidative dye precursor. Additional oxidative dye precursors, such as dimethylpiperazinium aminopyrazolopyridine and 2,3-diaminodihydropyrazolo pyrazolone dimethosulfonate, etc., may also optionally be included. The hair coloring base composition is combined with a developer composition to derive a ready-to-use hair coloring composition having an unusually low pH, in the range of about 6 to about 8. These unique hair coloring compositions impart long-lasting color to hair yet are gentle and non-irritating to the skin. They are useful for coloring all types of hair including hair of the head, eye lashes, eyebrows, and body, but are particularly useful for coloring facial hair (beard and mustache).

The hair coloring base compositions include:
2-methoxymethyl-P-phenylenediamine, and optionally one or more additional oxidative dye precursors;
less than 3 wt. %, preferably less than 2.5 wt. %, more preferably less than 2 wt. %, even more preferably less than 1.5 wt. % of one or more organic and/or mineral alkalizing agents, based on the total weight of the hair coloring base composition;
a combination of surfactants, water-soluble organic solvents, and water constituting at least 70 wt. %, preferably at least 75 wt. %, more preferably at least 80 wt. % of the hair coloring base composition, based on the total weight of the hair coloring base composition.

The pH of the hair coloring base compositions is typically from about 7 to about 10, preferably from about 7.5 to about 9.5, more preferably from about 8 to less than 9.5. A lower pH for the hair coloring base composition is useful for influencing the pH of a ready-to-use hair coloring compositions formed by combining the hair coloring base composition with a developer composition. The pH of the ready-to-use hair coloring compositions formed with the hair coloring base compositions is from about 6 to about 8, preferably from about 6.1 to about 7.9, more preferably from about 6.2 to about 7.8, which is lower than most ready-to-use hair coloring compositions.

Alkalizing agents have multiple roles in the coloring process, in addition to providing alkalinity to influence pH. Nonlimiting examples of organic alkalizing agents include monoethanolamine, diethanolamine, triethanolamine, monoisopropanolamine, diisopropanolamine, triisopropanolamine, N,N-dimethylethanolamine, 2-amino-2-methyl-1-propanol, 2-amino-2-methyl-1,3-propanediol, 3-amino-1,2-propanediol, 3-dimethylamino-1,2-propanediol, tris(hydroxymethyl)amino-methane, or mixtures thereof. Nonlimiting examples of mineral alkalizing agents include ammonia, ammonium carbonates, sodium carbonates, potassium carbonates, ammonium bicarbonates, sodium bicarbonates, potassium bicarbonates, ammonium hydroxides, sodium hydroxides, potassium hydroxides, or mixtures thereof. However, in various embodiments, the hair coloring base composition is free or essentially free from ammonia and/or ammonium ions, and/or ammonium hydroxide.

Surfactants can be nonionic, anionic cationic, amphoteric (zwitterionic) surfactants, or cationic. In various embodiments, the hair coloring base compositions include one or more nonionic surfactants. In further embodiments, the hair coloring base compositions include one or more nonionic surfactants and one of more anionic surfactants, for example, a fatty amide (nonionic surfactant) and an ether carboxylic acid (anionic surfactant).

The hair coloring base compositions are aqueous. In addition to water, however, the hair coloring compositions may include one or more water-soluble organic solvents. Nonlimiting examples of water-soluble organic solvents include glycerin, mono-alcohols, polyols (polyhydric alcohols), glycols, and mixture thereofs, e.g., glycerin, propylene glycol, butylene glycol, pentylene glycol, dipropylene glycol, hexylene glycol, ethanol, isopropanol, t-butyl alcohol, and mixture thereof.

Additional components useful in the hair coloring base compositions include fatty alcohols, surfactants, and/or water-soluble organic solvents. In certain embodiments, the fatty acids have from 12 to 24 carbon atoms. Nonlimiting examples include cetyl alcohol, stearyl alcohol, cetearyl alcohol, behenyl alcohol, lauryl alcohol, myristic or myristyl alcohol, arachidyl alcohol, lignoceryl alcohol, and mixtures thereof.

In various embodiments, the hair coloring base compositions include one or more reducing agents. Reducing agents are chemical species that lose an electron to another chemical species in a redox chemical reaction. Nonlimiting examples of reducing agents include ammonium bisulfite, ammonium sulfite, potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof. In certain embodiments, the hair coloring base compositions include thioglycolic acid, thiolactic acid, salts thereof, and mixtures thereof.

Further to the above, in various embodiments, the hair coloring base composition includes:
(a) 2-methoxymethyl-P-phenylenediamine, and optionally one or more additional oxidative dye precursors;
(b) one or more couplers;
(c) less than 3 wt. %, preferably less than 2.5 wt. %, more preferably less than 2 wt. %, even more preferably less than 1.5 wt. % of one or more organic and/or mineral alkalizing agents, based on the total weight of the hair coloring base composition;
(d) one or more fatty alcohols;
(e) one more surfactants;
(f) optionally one or more water-soluble organic solvents;
(g) water.
   wherein (e), (f), and (g) are in amounts such that a combined amount of (e), (f), and (g) constitutes at least 70 wt. %, preferably at least 75 wt. %, more preferably at least 80 wt. % of the hair coloring base composition; and
   all percentages by weight are based on the total weight of the hair coloring base composition.

In various embodiments, the hair coloring base compositions are free or essentially free from ammonia and/or ammonium ions and/or ammonium hydroxide.

The hair coloring base compositions are mixed with developer compositions to form ready-to-use hair coloring compositions having a pH of about 6 to about 8, preferably about 6.1 to about 7.9, or more preferably about 6.2 to about 7.8. For example, the hair coloring base compositions may be mixed with a developer composition in a weight ratio of about 5:1 to about 1:5, about 3:1 to about 1:3, or about 2:1 to about 1:2. The pH of the hair coloring base compositions is typically from about 7 to about 10, preferably from about 7.5 to about 9.5, more preferably from about 8 to less than 9.5. A lower pH for the hair coloring base composition is useful for influencing the pH of a ready-to-use hair coloring compositions formed by combining the hair coloring base composition with a developer composition.

The hair coloring base compositions and the developer compositions are separately contained prior to mixing. Thus, both compositions can be provided in a kit. Kits according to the instant disclosure include one or more hair coloring base compositions and one or more developer compositions, wherein the hair coloring base compositions and the developer compositions are separately contained. For example, the one or more hair coloring base compositions and the one or more developer compositions may be included in separate containers that are packaged together.

Methods for making a ready-to-use hair coloring compositions and methods for coloring hair with the compositions entail combining the hair coloring base composition with a developer composition to form a ready-to-use hair coloring composition. The ready-to-use hair coloring composition is then applied to hair for a period of time (for processing), for example, for about 1 to about 30 minutes, about 1 to about 20 minutes, or about 1 to about 15 minutes, about 1 to 10 minutes, or amount 1 to 5 minutes. After the period of time has lapsed, the hair coloring compositions may be rinsed or washed from the hair exposing the newly colored hair. The newly colored hair durably retains its changed color, suffers little or no undesirable chemical damage, and the underlying skin is not subjected to burning and stinging associated with various oxidative coloring methods.

### BRIEF DESCRIPTION OF THE DRAWINGS

Implementations of the present technology will now be described, by way of example, with reference to the attached figures, wherein:

FIG. 1 is a graph showing the color retention of 90% grey hair treated with an inventive composition verses hair treated with two comparative compositions; and

FIG. 2 is a graph showing the color retention of 90% permed hair treated with an inventive composition verses hair treated with two comparative compositions.

The figures and the data they represent are nonlimiting and are provided merely to illustrate certain aspects of the technology.

### DETAILED DESCRIPTION

Individuals often seek to permanently change the color of hair using oxidative dying procedures. Common complaints associated with these procedures include inadequate color deposition, damage to the hair and/or underlying skin, and loss of color from the hair over time. During the coloring process, hair coloring compositions often come into contact with underlying skin. This can temporarily discolor the underlying skin, cause discomfort, and in some instances even damage the skin. Over time, the initial color imparted to the hair can fade. As color fades, the hair can appear less vibrant, exhibit unwanted color tones, and look unkept.

The hair coloring compositions and method of the instant case address the concerns discussed above. They are gentle to the hair and underlying skin yet provide quick and powerful color deposition to the hair that is long-lasting (durable). The hair coloring compositions are suitable for all types of hair. However, due to their robust coloring ability and gentle nature, they are particularly well-suitable for coloring facial hair (e.g., beards and mustache), which tends to be coarser than other hair.

Different parts of the body grow different types of hair. “Lanugo hair” is the covering of fine hair which covers most newborn babies. It tends to fall out soon before or just after birth. “Vellus hair” is short, fine, and lightly colored. It develops over the body during childhood, except for the lips, palms, soles, back of the ears, in the navel, etc. These hairs sprout from follicles which are not connected to an oil gland. “Terminal hair grows primarily on the head. It is long, thick, and darker than hair elsewhere on the body. When a person enters puberty, terminal hair replaces vellus hair on the pubic area and in the armpits. Men in particular may also develop terminal hair in place of vellus hair on the chest, limbs, feet, back, and face. When it develops in puberty/adolescence, it is called androgenic hair.

The hair on the scalp, like that on the face, grows in three steps, but the hair on the face has a much shorter growing phase. These steps include the active growth phase (anagen), the transitional phase (catagen—when growth stops) and the falling out phase (telogen). On the scalp, this cycle lasts for years; on the face it is a matter of months. Beard and mustache hair (androgenic hair) can be very different than hair grown on the scalp, or elsewhere on the body. Androgenic facial hair tends to be thicker with a wirier texture and can exhibit a different color than scalp hair. An individual may retain a full head of colored hair into middle age, yet the beard may grow partially or completely grey. Scalp hair may be straight, yet androgenic facial hairs grow thicker and curly due in part to the different shape of the follicles. Follicles on the face are much more sensitive to androgens like testosterone, which can cause follicles to twist and grow curly hair. The skin under androgenic facial hair is also different from skin of the scalp.

As noted previously, hair coloring compositions for permanently altering the color of hair typically rely on a combination of two parts, a hair coloring base composition, and a developer composition. The hair coloring base composition includes oxidative dye precursors, such as 2-methoxymethyl-P-phenylenediamine, and the developer composition includes oxidizing agents, like hydrogen peroxide. The two parts are mixed to form a ready-to-use hair coloring composition.

Oxidative dye precursors are typically colorless or weakly colored compounds, which, when combined with oxidizing agents, transition to provide colored species via a process of oxidative condensation. The shades obtained with oxidative dye precursors may be varied by combining them with one or more couplers. Couplers include, for example, aromatic meta-diamines, meta-aminophenols, meta-diphenols, and certain heterocyclic compounds, such as indole compounds.

The oxidizing agent(s) employed in permanent dyeing compositions may degrade the melanin of the hair, which, depending on the nature of the oxidizing agent, may lead to less pronounced lightening of the fibers. A common oxidizing agent used in hair lightening and coloring is hydrogen peroxide. Nonetheless, peroxygenated salts, such as persulfates, may be used, often in conjunction of hydrogen peroxide.

In general, hair coloring compositions are alkaline, having a high pH of about 9 and higher, and may generally require the presence of an alkalizing agent such as ammonia or an ammonia gas-generating compound and/or an amine or ammonium-based compound in amounts sufficient to achieve the desired alkalinity. The alkalizing agents help activate the oxidizing agents and cause the hair shaft to swell, thus allowing the small oxidative dye precursor molecules to penetrate the cuticle and cortex before the oxidation condensation process is completed. The resulting larger-sized colored complexes from the oxidative reaction are then trapped inside the hair fiber, permanently altering the color of the hair.

In various embodiments, the hair coloring base compositions of the instant disclosure include:
(a) 2-methoxymethyl-P-phenylenediamine, and optionally one or more additional oxidative dye precursors;
(b) one or more couplers;
(c) less than 3 wt. %, preferably less than 2.5 wt. %, more preferably less than 2 wt. %, even more preferably less than 1.5 wt. % of one or more organic and/or mineral alkalizing agents, based on the total weight of the hair coloring base composition;
(d) about 0.1 to about 10 wt. % of one or more fatty alcohols, for example, fatty alcohols having from 12 to 24 carbon atoms;
(e) about 15 to about 40 wt. % of one or more surfactants;
(f) about 10 to about 40 wt. % of one or more water-soluble organic solvents;
(g) about 25 to about 60 wt. % water;
   wherein (e), (f), and (g) are in amounts such that a combined amount of (e), (f), and (g) constitutes at least 70 wt. % of the hair coloring base composition; and
   all percentages by weight are based on the total weight of the hair coloring base composition.

In various embodiments, the hair coloring base compositions are free for essentially free rom ammonia and/or ammonium ions and/or ammonium hydroxide.

The pH of the hair coloring base composition will vary. However, the pH is typically such that when mixed with a developer composition to form a ready-to-use hair coloring composition, the ready-to-use hair coloring composition has a desired pH, e.g., a pH from about 6 to about 8, preferably about 6.1 to about 7.9, more preferably from about 6.2 to about 7.8. Developer composition are typically acidic, for example, having a pH of 2 to about 6. Therefore, to derive a ready-to-use hair coloring composition by mixing the developer composition with the hair coloring base composition, the hair coloring base composition should have a low pH, for example from about 7 to about 10, preferably from about 7.5 to about 9.5, more preferably from about 8 to less than 9.5. The pH of the hair coloring base composition, the pH of the developer composition, and the mixing ratio of hair coloring base composition to developer composition can influence the final pH of the ready-to-use hair coloring composition.

In various embodiments, the pH of the hair coloring base composition is from about 7 to about 10, about 7.5 to about 10, about 8 to about 10, from about 8.5 to about 10, from about 7 to about 9.5, from about 7.5 to about 9.5, from about 8 to about 9.5, from about 8.5 to about 9.5, from about 7 to about 9, from about 7.5 to about 9, or from about 8 to about 9.

In various embodiments, the hair coloring base compositions are free or essentially free from resorcinol and/or resorcinol derivatives. The hair color altering compositions may, in some embodiments, be free or substantially free of para-phenylenediamines (other than 2-methoxymethyl-P-phenylenediamine), resorcinol, and/or resorcinol derivatives. In some embodiments, the hair color altering compositions may be free or substantially free of one or more of mineral oil, ammonia, ammonium hydroxide, ammonium thiolactate, para-phenylenediamines (other than 2-methoxymethyl-P-phenylenediamine), resorcinol, and/or resorcinol derivatives. In other embodiments, the hair color altering compositions may be free or substantially free of mineral oil, ammonia, ammonium hydroxide, ammonium thiolactate, para-phenylenediamines (other than 2-methoxymethyl-P-phenylenediamine), resorcinol, and resorcinol derivatives.

As noted, the hair coloring base compositions of the instant disclosure are combinable with developer composition to form a ready-to-use hair coloring composition having a pH from about 6 to about 8, preferably about 6.1 to about 7.9, or more preferably about 6.2 to about 7.8. The final pH of a ready-to-use hair coloring composition will depend on the content, pH, etc., of the hair coloring base composition. In various embodiments, the hair coloring base compositions of the instant disclosure are such that they form a ready-to-use hair coloring composition having a pH from about 6 to about 8, about 6.1 to about 7.9, or about 6.2 to about 7.8, upon mixing with a developer composition comprising hydrogen peroxide in a weight ratio about 1:5 to about 5:1 (hair coloring composition:developer composition).

Furthermore, the total amount of alkalizing agent (e.g., monoethanolamine) in the hair coloring base composition such that when mixed with a developer composition to form a ready-to-use hair coloring composition, the total amount of alkalizing agent in the ready-to-use coloring composition is less than 2 wt. %, preferably less than 1.5 wt. %, more preferably less than 1 wt. %

For example, the hair coloring base compositions are such that they form a ready-to-use hair coloring composition having a pH from about 6 to about 8, about 6.1 to about 7.9, or about 6.2 to about 7.8, upon mixing with a developer composition comprising hydrogen peroxide in a weight ratio about 1:1 (hair coloring composition:developer composition), wherein the developer composition consists of:
(a) about 7.5 wt. % hydrogen peroxide;
(b) about 82 wt. % water;
(c) about 0.5 wt. % glycerin;
(d) about 2.3 wt. % of cetearyl alcohol;
(e) about 0.9 wt. % of trideceth-2 carboxamide MEA;
(f) about 0.6 wt. % ceteareth-25; and
   wherein all percentages by weight for components of the developer composition are based on the total weight of the developer composition.

The hair coloring base compositions include 2-methoxymethyl-P-phenylenediamine, which is an oxidative dye precursor, and optionally one or more additional oxidative dye precursors. Oxidative dye precursors are also referred to as “primary intermediates” or “oxidation bases.” Oxidative dye precursors are often colorless or weakly colored compounds, which, when combined with oxidizing products, reactive via oxidative condensation to provide colored species. Non-limiting examples of oxidative dye precursors include aromatic diamines, polyhydric phenols, amino phenols, and derivatives of these compounds, such as, for example, N-substituted derivatives of the amines, and ethers of the phenols, ortho- or para-aminophenols, ortho- or para-phenylenediamines, double bases, heterocyclic bases, and the acid addition salts thereof. More specific nonlimiting examples include dimethylpiperazinium aminopyrazolopyridine, 2,3-diaminodihydropyrazolo pyrazolone dimethosulfonate, p-phenylene diamine, 2,5-diaminotoluene, N,N-bis(2-hydroxymethyl)-p-phenylene diamine, paminophenol, salts thereof, etc.

In certain embodiments, in addition to the 2-methoxymethyl-P-phenylenediamine, the hair coloring base composition includes one or more oxidative dye precursors chosen from dimethylpiperazinium aminopyrazolopyridine, 2,3-diaminodihydropyrazolo pyrazolone dimethosulfonate, salts thereof, and combinations thereof.

A more exhaustive but nonlimiting list of useful oxidative dye precursors is described later, under the heading “Oxidative Dye Precursors.”

The total amount of the one or more oxidative dye precursors, including 2-methoxymethyl-P-phenylenediamine will vary depending on the desired color and intensity of the hair to be treated. In various embodiments, the total amount of the one or more oxidative dye precursors, including 2-methoxymethyl-P-phenylenediamine, is from about 0.001 to about 12 wt. %, about 0.001 to about 10 wt. %, about 0.001 to about 8 wt. %, about 0.001 to about 5 wt. %, about 0.01 to about 10 wt. %, about 0.01 to about 8 wt. %, about 0.01 to about 6 wt. %, or about 0.01 to about 5 wt. %, about 0.1 to about 12 wt. %, about 0.1 to about 10 wt. %, about 0.1 to about 8 wt. %, about 0.1 to about 6 wt. %, about 0.1 to about 5 wt. %, about 0.5 to about 12 wt. %, about 0.5 to about 10 wt. %, about 0.5 to about 8 wt. %, about 0.5 to about 6 wt. %, or about 0.5 to about 5 wt. %, based on the total weight of the hair coloring base composition.

With respect to 2-methoxymethyl-P-phenylenediamine individually, in various embodiments, it is present in an amount of about 0.001 to about 10 wt. %, about 0.001 to about 8 wt. %, about 0.001 to about 5 wt. %, about 0.001 to about 4 wt. %, about 0.01 to about 10 wt. %, about 0.01 to about 8 wt. %, or about 0.01 to about 5 wt. %, about 0.01 to about 4 wt. %, about 0.1 to about 10 wt. %, about 0.1 to about 8 wt. %, about 0.1 to about 5 wt. %, about 0.1 to about 4 wt. %, about 0.1 to about 3.5 wt. %, about 0.1 to about 3 wt. %, about 0.5 to about 8 wt. %, or about 0.5 to about 5 wt. %, about 0.5 to about 4 wt. %, about 0.5 to about 3.5 wt. %, or about 0.5 to about 3 wt. %, based on the total weight of the hair coloring base composition

The colors obtained using oxidative dye precursors can be varied by combining them with one or more couplers. Thus, in certain embodiments, the hair coloring base compositions of the instant disclosure include one or more couplers. Non-limiting examples of couplers include aromatic meta-diamines, meta-aminophenols, meta-diphenols, and certain heterocyclic compounds, such as indole compounds. A more exhaustive but non-limiting list of couplers that may be included in the hair coloring base compositions is provided later, under the heading “Couplers.” The variety of molecules used as oxidative dye precursors and couplers allow for a wide range of colors to be obtained.

The total amount of couplers, if present, will vary depending on the desired color of the hair to be treated. In various embodiments, the hair coloring base composition includes about 0.0001 to about 12 wt. %, based on the total weight of the hair coloring base composition. In some instances, the total amount of the one or couplers is about 0.001 to about 12 wt. %, about 0.001 to about 10 wt. %, about 0.001 to about 5 wt. %, about 0.001 to about 3 wt. %, about 0.01 to about 10 wt. %, about 0.01 to about 8 wt. %, about 0.01 to about 5 wt. %, or about 0.01 to about 3 wt. %, about 0.1 to about 10 wt. %, about 0.1 to about 8 wt. %, about 0.1 to about 5 wt. %, about 0.1 to about 4 wt. %, or about 0.1 to about 3 wt. %, based on the total weight of the hair coloring base composition.

The weight ratio of the total amount of the oxidative dye precursors (including the 2-methoxymethyl-P-phenylenediamine) to the total amount of the one or more couplers in the hair coloring base composition may be from about 1:1 to about 5:1, about 1:1 to about 4:1, or about 1:1 to about 3:1, about 1:1 to about 2:1, or about 1:1 to about 1.5:1.

In addition to oxidative dye precursors, and optionally couplers, the hair coloring base composition, in certain embodiments, includes one or more direct dyes, pigments, and/or mixtures thereof. The total amount of the one or more direct dyes, pigments, and mixtures thereof, if present, will vary. Nonetheless, in certain embodiments the total amount of the one or more direct dyes, pigments, and/or mixtures thereof, if present, is from about 0.01 to about 5 wt. %, about 0.01 to about 3 wt. %, about 0.01 to about 2 wt. %, about 0.1 to about 5 wt. %, about 0.1 to about 3 wt. %, about 0.1 to about 2 wt. %, or about 0.1 to about 1 wt. %, based on the total weight of the hair coloring base composition.

Alkalizing agents in the hair coloring base composition typically have multiple roles in the coloring process. For instance, the one or more alkalizing agents are helpful for obtaining a desired pH. In addition, the one or more alkalizing agents help cause the hair shaft to swell, allowing the small oxidative dye precursor molecules to penetrate the cuticle and cortex more easily. Also, the alkalizing agents can activate one or more oxidizing agents of the developer composition and contribute to the oxidation condensation process. Non-limiting examples of alkalizing agents include ammonia, ammonium hydroxide, ammonium carbonate, ammonium chloride, ammonium sulfate, ammonium nitrate, ammonium phosphate, ammonium acetate, ammonium hydrogen carbonate, ammonium carbamate, percarbonate salts, alkanolamines (such as monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol), guanidium salts, alkali metal hydroxides (such as sodium hydroxide), alkali metal carbonates, and a mixture thereof.

The hair coloring base compositions according to the instant disclosure may comprise one or more alkalizing agents. According to various embodiments, the alkalizing agent may comprise at least one organic alkalizing agent and/or at least one mineral alkalizing agent.

In certain embodiments, the alkalizing agent comprises at least one organic alkalizing agent chosen from monoethanolamine, diethanolamine, triethanolamine, monoisopropanolamine, diisopropanolamine, triisopropanolamine, N,N-dimethylethanolamine, 2-amino-2-methyl-1-propanol, 2-amino-2-methyl-1,3-propanediol, 3-amino-1,2-propanediol, 3-dimethylamino-1,2-propanediol, tris(hydroxymethyl)amino-methane, or mixtures thereof.

In further embodiments, the alkalizing agent comprises at least one mineral alkalizing agent chosen from ammonia, ammonium carbonates, sodium carbonates, potassium carbonates, ammonium bicarbonates, sodium bicarbonates, potassium bicarbonates, ammonium hydroxides, sodium hydroxides, potassium hydroxides, or mixtures thereof. In some embodiments, the alkaline component comprises ammonia and/or ammonium hydroxide.

In some embodiments, the alkalizing agent comprises at least one organic alkalizing agent and is free or substantially free of mineral alkalizing agents. For example, the alkalizing agent may comprise less than about 0.5%, less than about 0.4%, less than about 0.3%, less than about 0.2%, less than about 0.1%, or less than about 0.05% of mineral alkalizing agents. In certain embodiments, the alkalizing agent comprises at least one organic alkalizing agent and is free or substantially free of ammonia and/or ammonium ions, and/or ammonium-based compounds. In various embodiments, the alkalizing agent comprises monoethanolamine. In further embodiments, the alkalizing agent comprises monoethanolamine and is free or substantially free of ammonia and/or ammonium ions and/or ammonium-based compounds. An “ammonium-based compound” in the context of the instant disclosure is a compound which produces ammonia when in the composition at a particular pH. Examples of such compounds include ammonia and compounds which may be added as ammonium hydroxide and ammonium salts (e.g., simple salts). As ammonium salts, mention may be made of inorganic ammonium salts such as ammonium carbonate, ammonium bicarbonate, ammonium chloride, ammonium nitrate, ammonium sulfate, ammonium phosphate; organic ammonium salts such as ammonium formate, ammonium acetate, and tetramethylammonium hydroxide; and mixtures thereof,

The total amount of the one or more alkalizing agents in the hair coloring base compositions will vary. Nonetheless, in various embodiments, the hair coloring base composition includes less than about 3 wt. % of alkalizing agents, based on the total weight of the hair coloring base composition. In further embodiments, the hair coloring base composition includes less than 3 wt. %, less than 2.5 wt. %, less than 2.2 wt. %, less than 2 wt. %, less than 1.8 wt. %, less than 1.6 wt. %, less than 1.5 wt. %, less than 1.3 wt. %, less than 1.1 wt. %, less than 1 wt. %, based on the total weight of the hair coloring base composition.

The types of reducing agents that may be included in the hair coloring base compositions can vary. Nonlimiting examples of reducing agents include ammonium bisulfite, ammonium sulfite, potassium metabisulfite, potassium sulfite, sodium hydrosulfite, sodium metabisulfite, sodium sulfite, sodium bisulphite, thioglycolic acid, thiolactic acid, dehydroascorbic acid, salts thereof, and mixtures thereof. The salts referred to throughout the disclosure may include salts having a counter-ion such as an alkali metal, alkaline earth metal, or ammonium counterion. This list of counterions, however, is non-limiting. In various embodiments, preferred reducing agents include thiolactic acid, glycolic acid, salts thereof (e.g., ammonium thiolactate), and combinations thereof.

In various embodiments, the one or more reducing agents may be from thiols such as thioglycolic acid or a salt thereof, thiolactic acid or a salt thereof, 3-mercaptopropionic acid, thiomalic acid, 2,3-dimercaptosuccinic acid, cysteine, N-glycyl-L-cysteine, L-cysteinylglycine and also esters and salts thereof, thioglycerol, cysteamine and C₁-C₄ acyl derivatives thereof, N-mesylcysteamine, N-acetylcysteine, N-mercaptoalkylamides of sugars such as N-(mercapto-2-ethyl) gluconamide, pantetheine, N-(mercaptoalkyl)-Ω-hydroxyalkylamides, N-mono- or N,N-dialkylmercapto-4-butyramides, aminomercaptoalkyl amides, N-(mercaptoalkyl)succinamic acids and N-(mercaptoalkyl)succinimides, alkylamino mercaptoalkyl amides, mercaptoalkylamino amides, N-mercaptoalkylalkanediamides, ammonium bisulfite, ammonium sulfite, potassium metabisulfite, potassium sulfite, sodium hydrosulfite, sodium metabisulfite, sodium sulfite, sodium bisulphite, and a mixture thereof. In further embodiments, the hair coloring base composition include one or more reducing agents chosen from thioglycolic acid, thiolactic acid, 3-mercaptopropionic acid, salts thereof, and a combination thereof.

The reducing agent may also be chosen from hydrides such as sodium or potassium borohydride or alkali metal or alkaline-earth metal sulfites or bisulfites; or alternatively from phosphorus derivatives such as phosphines or phosphites. In various embodiments, the one or more reducing agents are chosen from ammonium bisulfite, ammonium sulfite, potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof.

The total amount of the one more reducing agents in the hair coloring base composition may vary but is typically about 0.01 to about 10 wt. %, based on the total weight of the composition. The total amount of the one or more reducing agents may be about 0.01 to about 8 wt. %, about 0.01 to about 5 wt. %, about 0.1 to about 10 wt. %, about 0.1 to about 8 wt. %, about 0.1 to about 5 wt. %, about 0.1 to about 3 wt. %, or about 0.1 to about 2 wt. %, based on the total weight of the hair coloring base composition.

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. Nonlimiting examples of solid fatty alcohols include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In certain embodiments, the one or more fatty alcohols have from 12 to 24 carbon atoms. Specific nonlimiting examples include cetyl alcohol, stearyl alcohol, cetearyl alcohol, behenyl alcohol, lauryl alcohol, myristic or myristyl alcohol, arachidyl alcohol, lignoceryl alcohol, or mixtures thereof. Preferably, the cosmetic composition includes one or more fatty alcohols chosen from cetyl alcohol, stearyl alcohol, behenyl alcohol, oleyl alcohol, and mixtures thereof such as cetylstearyl or cetearyl alcohol.

The liquid fatty alcohols, in particular those containing C10-C34, preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched alkyl group or an alkenyl group (comprising at least one C12-C24 double bond C═C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof.

In various embodiments, the hair coloring base composition includes one or more fatty alcohols chosen from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the hair coloring base compositions preferably includes at least oleyl alcohol.

The total amount of the one or more fatty alcohols, if present, will vary. In various embodiments, the hair coloring base composition includes from about 0.1 to about 10 wt. %, about 0.1 to about 8 wt. %, about 0.1 to about 5 wt. %, about 0.5 to about 10 wt. %, about 0.5 to about 8 wt. %, about 0.5 to about 5 wt. %, or about 0.5 to about 3 wt. %, based on the total weight of the hair coloring base composition.

In various embodiments, the hair coloring base composition includes one or more surfactants, for example, one or more anionic surfactants, nonionic surfactants, amphoteric surfactants (zwitterionic surfactants), cationic surfactants and/or a mixture thereof. In a preferred embodiment, the hair coloring base composition includes at least one or more nonionic surfactants and optionally, or preferably, one or more anionic surfactants. In certain embodiments, the hair coloring base compositions is preferably free or essentially free from cationic surfactants.

The total amount of the one or more surfactants can vary but is typically about 1 to about 40 wt. %, based on the total weight of the hair coloring base composition. The total amount of the one or more surfactants may be about 1 to about 35 wt. %, about 1 to about 30 wt. %, about 5 to about 40 wt. %, about 5 to about 32 wt. %, about 10 to about 40 wt. %, about 10 to about 35 wt. %, about 10 to about 32 wt. %, about 15 to about 40 wt. %, about 15 to about 35 wt. %, about 15 to about 32 wt. %, about 20 to about 40 wt. %, about 20 to about 32 wt. %, about 20 to about 32 wt. %, or about 25 to about 32 wt. %, based on the total weight of the hair coloring base composition.

If various embodiments, the total amount of the one or more surfactants in the hair coloring base composition is at least 10 wt. %, at least 12 wt. %, at least 15 wt. %, at least 18 wt. %, at least 20 wt. %, at least 21 wt. %, at least 22 wt. %, at least 23 wt. %, or at least 24 wt. %, in each case having an optional maximum amount of up to 30, 35, or 40 wt. %, based on the total weight of the hair coloring base composition.

In various embodiments, the hair coloring base composition includes one or more nonionic surfactants. Nonlimiting examples of nonionic surfactants include oxyethylenated amides, oxyethylenated fatty alcohols, and block-copolymer (polycondensate) surfactants of ethylene oxide and of propylene oxide, and a mixture thereof. In a preferred embodiment, the hair coloring base composition includes PEG-4 rapeseedamide (an oxyethylenated amide), deceth-3 (an oxyethylenated fatty alcohol), poloxamer 338 (block-copolymer (polycondensate) surfactants of ethylene oxide and of propylene oxide), or a combination thereof.

Non-limiting examples of nonionic oxyethylenated amides are those of the following formula:

R—[(OCH2CH2)n-OCH2]ₚ—CO—N(R′)—(CH2CH2O)ₙ′H
in which:
p denotes 0 or 1,
n denotes a number ranging from 1 to 10 and preferably from 1 to 6,
n′ denotes a number ranging from 1 to 100 and preferably from 1 to 60,
R′ denotes a hydrogen atom or a CH2CH2OH radical and preferably a hydrogen atom, and
R denotes a C10-C30 and preferably C12-C22 alkyl or alkenyl radical.

Examples of these compounds include AMIDET A15 sold by the company Kao (INCI name: Trideceth-2 carboxamide MEA), ETHOMID HP 60 sold by the company Akzo Nobel (INCI name: PEG-50 Hydrogenated Palmamide) and AMIDET N sold by the company Kao (INCI name: PEG-4 Rapeseedamide).

In some cases, the hair coloring base compositions includes at least rapeseed amide oxyethylenated with 4 oxyethylene units (PEG-4 rapeseedamide).

Non-limiting examples of fatty alcohols include saturated or unsaturated and linear or branched alcohols comprising from 6 to 30 carbon atoms and preferably from 8 to 30 carbon atoms, for instance, cetyl alcohol, isostearyl alcohol, stearyl alcohol and the mixture thereof (cetylstearyl alcohol), octyldodecanol, 2-butyloctanol, 2-hexyldecanol, 2-undecylpentadecanol, oleyl alcohol, linolenyl alcohol, ricinoleyl alcohol, undecylenyl alcohol and linoleyl alcohol, and mixtures thereof.

Non-limiting examples of oxyethylenated fatty alcohols include those comprising less than 10 OE units, preferably chosen from oxyethylenated derivatives of saturated or unsaturated, linear or branched, preferably linear, C₈-C₃₀ and preferably C₁₂-C₂₂ fatty alcohols, for instance cetyl alcohol, oleyl alcohol, oleocetyl alcohol, lauryl alcohol, behenyl alcohol, cetearyl alcohol, stearyl alcohol and isostearyl alcohol, and mixtures thereof.

As oxyethylenated fatty alcohols comprising less than 10 OE units, mention may be made of oxyethylenated fatty alcohols comprising from 2 to 8 and preferably from 2 to 6 OE units, for instance products of addition of ethylene oxide and lauryl alcohol, for instance lauryl alcohol 2 OE (CTFA name: laureth-2), products of addition of ethylene oxide and stearyl alcohol, for instance stearyl alcohol 2 OE (CTFA name: steareth-2), products of addition of ethylene oxide and decyl alcohol, for instance decyl alcohol 3 OE (CTFA name: deceth-3), decyl alcohol 5 OE (CTFA name: deceth-5), products of addition of ethylene oxide and oleocetyl alcohol, for instance oleocetyl alcohol 5 OE (CTFA name: oleoceteth-5), and mixtures thereof. In some instances, deceth-3 may be particularly useful.

Furthermore, non-limiting examples of oxyethylenated fatty alcohols having an average degree of ethoxylation of 2 to 29 are, for example, laureth-2, oleth-2, ceteareth-2, laneth-2, laureth-3, oleth-3, ceteareth-3, laureth-4, oleth-4, ceteareth-4, laneth-4, laureth-5, oleth-5, ceteareth-5, laneth-5, deceth-4, deceth-7, laureth-7, oleth-7, coceth-7, ceteth-7, ceteareth-7, C11-15 pareth-7, laureth-9, oleth-9, ceteareth-9, laureth-10, oleth-10, beheneth-10, ceteareth-10, laureth-12, ceteareth-12, trideceth-12, ceteth-15, laneth-15, ceteareth-15, laneth-16, ceteth-16, oleth-16, steareth-16, oleth-20, ceteth-20, ceteareth-20, laneth-20, steareth-21, ceteareth-23, ceteareth-25, ceteareth-27, and a mixture thereof.

In some cases, the hair coloring base composition includes both at least one nonionic surfactant chosen from oxyethylenated amide and at least one nonionic surfactant chosen from oxyethylenated (OE) fatty alcohol comprising less than 10 OE units, that may be chosen among those described above.

Furthermore, the hair coloring base composition may include one or more nonionic surfactants that is a block-copolymer (polycondensate) surfactant of ethylene oxide and of propylene oxide. The block-copolymer (polycondensate) surfactant of ethylene oxide and of propylene oxide may have a weight-average molecular weight ranging from 1000 to 20000, better from 1500 to 19000, from 2000 to 18000, or from 4000 to 17000.

Mention may be made, as block-copolymer (polycondensate) surfactant of ethylene oxide and of propylene oxide which may be used, of the polyethylene glycol/polypropylene glycol/polyethylene glycol triblock polycondensates sold under the “SYNPERONIC” names, such as “SYNPERONIC PE/F32” (INCI name: Poloxamer 108), “SYNPERONI. PE/F108” (INCI name: Poloxamer 338), “SYNPERONIC PE/L44” (INCI name: Poloxamer 124), “SYNPERONIC PE/L42” (INCI name: Poloxamer 122), “SYNPERONIC PE/F127” (INCI name: Poloxamer 407), “SYNPERONIC PE/F88” (INCI name: Poloxamer 238) or “SYNPERONIC PE/L64” (INCI name: Poloxamer 184), by Croda or also “LUTROL F68” (INCI name: Poloxamer 188), sold by BASF. In some instances, Poloxamer 338 may be particularly useful.

A more exhaustive list of useful nonionic surfactants that may be included in the hair coloring base composition is provided later, under the heading “Nonionic Surfactants.”

The total amount of the one or more nonionic surfactants in the hair coloring base composition can vary but is typically about 1 to about 40 wt. %, based on the total weight of the hair coloring base composition. The total amount of the one or more nonionic surfactants may be about 1 to about 35 wt. %, about 1 to about 30 wt. %, about 5 to about 40 wt. %, about 5 to about 32 wt. %, about 10 to about 40 wt. %, about 10 to about 35 wt. %, about 10 to about 32 wt. %, about 15 to about 40 wt. %, about 15 to about 35 wt. %, about 15 to about 32 wt. %, about 20 to about 40 wt. %, about 20 to about 32 wt. %, about 20 to about 32 wt. %, or about 25 to about 32 wt. %, based on the total weight of the hair coloring base composition.

If various embodiments, the total amount of the one or more nonionic surfactants in the hair coloring base composition is at least 10 wt. %, at least 12 wt. %, at least 15 wt. %, at least 18 wt. %, at least 20 wt. %, at least 21 wt. %, at least 22 wt. %, at least 23 wt. %, or at least 24 wt. %, in each case having an optional maximum amount of up to 30, 35, or 40 wt. %, based on the total weight of the hair coloring base composition.

In various embodiments, the hair coloring base composition includes one or more anionic surfactants. For instance, in certain embodiments, the hair coloring base composition includes one or more nonionic surfactants, one or more anionic surfactants, or a mixture thereof. In a preferred embodiment, the hair coloring base composition includes one or more nonionic surfactants and one or more anionic surfactants. The hair coloring base composition may also optionally include one or more amphoteric surfactants, one or more cationic surfactants, or a combination thereof. Nonetheless, in certain embodiments, the hair coloring base composition is free or essentially free from cationic surfactants and/or free or essentially free from amphoteric surfactants.

Nonlimiting examples of anionic surfactants include alkyl carboxylic acids, alkyl ether carboxylic acids, alkyl phosphates, alkyl ether phosphates, alkyl sulfates, alkyl ether sulfates, alkyl sulfonates, alkyl ether sulfonates, and salts thereof. In certain embodiments, the hair coloring base composition includes one or more alkyl ether carboxylic acids. Nonlimiting examples of alkyl ether carboxylic acids include ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, and mixtures and/or salts thereof.

The total amount of the one or more anionic surfactants, if present, will vary. In certain embodiments, the hair coloring base composition includes about 0.1 to about 15 wt. % of the one or more anionic surfactants, based on the total weight of the hair coloring base composition. In further embodiments, the hair coloring base composition includes about 0.1 to about 12 wt. %, about 0.1 to about 10 wt. %, about 0.1 to about 8 wt. %, about 0.1 to about 6 wt. %, about 0.5 to about 15 wt. %, about 0.5 to about 12 wt. %, about 0.5 to about 10 wt. %, about 0.5 to about 8 wt. %, about 0.5 to about 6 wt. %, about 1 to about 15 wt. %, about 1 to about 12 wt. %, about 1 to about 10 wt. %, about 1 to about 8 wt. %, about 2 to about 10 wt. %, about 3 to about 8 wt. %, or about 4 to about 7 wt. %, based on the total weight of the hair coloring base composition.

In various embodiments, the hair coloring base composition includes one or more water-soluble organic solvents (or simply “water-soluble solvents”). The term “water-soluble organic solvent” (or “water-soluble solvent”) is interchangeable with the term “water-miscible solvent” and means an organic compound that is liquid at 25° C. and at atmospheric pressure (760 mmHg), and it has a solubility of at least 50% in water under these conditions. In certain embodiments, the one or more water-soluble organic solvents have a solubility of at least 60%, 70%, 80%, or 90% in water at 25° C. and at atmospheric pressure (760 mmHg). Non-limiting examples of water-soluble organic solvents include glycerin, alcohols (for example, C₁₋₃₀, C₁₋₁₅, C₁₋₁₀, or C₁₋₄ alcohols), polyols, glycols, and a mixture thereof. In certain embodiments, the one or more water-soluble organic solvents are chosen from alcohols such as ethyl alcohol, isopropyl alcohol, propyl alcohol, benzyl alcohol, and phenylethyl alcohol, or glycols or glycol ethers such as monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Further non-limiting but useful examples of water-soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In various embodiments, the hair coloring base composition includes one or more water-soluble organic solvents chosen from glycols, C₁₋₄ alcohols, glycerin, and a mixture thereof; preferably the water-soluble organic solvent is chosen from caprylyl glycol, glycerin, ethanol, isopropyl alcohol, dipropylene glycol, propylene glycol, hexylene glycol, caprylyl glycol, propylene glycol, glycerin, ethanol, and a mixture thereof.

In certain embodiments, the hair coloring base composition includes one or more polyhydric alcohols. Nonlimiting examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The total amount of the one or more water-soluble organic solvents will vary. In various embodiments, the hair coloring base composition includes about 1 to about 50 wt. % of the one or more water-soluble organic solvents, based on the total weight of the hair coloring base composition. In further embodiments, the hair coloring base composition includes about 1 to about 45 wt. %, about 1 to about 40 wt. %, about 1 to about 30 wt. %, about 1 to about 25 wt. %, about 5 to about 50 wt. %, about 5 to about 40 wt. %, about 5 to about 30 wt. %, about 5 to about 25 wt. %, about 10 to about 50 wt. %, about 10 to about 40 wt. %, about 10 to about 30 wt. %, or about 10 to about 25 wt. %, about 15 to about 50 wt. %, about 15 to about 40 wt. %, about 15 to about 30 wt. %, about 15 to about 25 wt. %, or about 18 to about 23 wt. % of the one or more water-soluble organic solvents, based on the total weight of the hair coloring base composition.

If various embodiments, the total amount of the one or more water-soluble organic solvents in the hair coloring base composition is at least 10 wt. %, at least 12 wt. %, at least 15 wt. %, at least 18 wt. %, at least 20 wt. %, in each case having an optional maximum amount of up to 25, 30, 35, or 40 wt. %, based on the total weight of the hair coloring base composition.

The hair coloring base composition includes varying amounts of water. The amount of water may vary depending on the desired consistency of the product, the amount of water (if any) in a developer composition with which the hair coloring base composition will be mixed, etc. In various embodiments, the hair coloring base composition includes about 5 to about 75 wt. % of water, based on the total weight of the hair coloring base composition. In further embodiments, the total amount of water in the hair coloring base composition is from about 5 to about 70 wt. %, about 5 to about 65 wt. %, about 5 to about 60 wt. %, about 5 to about 50 wt. %, about 10 to about 75 wt. %, about 10 to about 70 wt. %, about 10 to about 60 wt. %, about 10 to about 50 wt. %, about 15 to about 75 wt. %, about 15 to about 70 wt. %, about 15 to about 60 wt. %, about 15 to about 50 wt. %, about 20 to about 75 wt. %, about 20 to about 70 wt. %, about 20 to about 60 wt. %, about 20 to about 50 wt. %, about 25 to about 60 wt. %, about 25 to about 55 wt. %, about 30 to about 60 wt. %, about 30 to about 55 wt. %, about 30 to about 50 wt. %, about 35 to about 60 wt. %, about 35 to about 55 wt. %, or about 25 to about 50 wt. %, based on the total weight of the hair coloring base composition.

If various embodiments, the total amount of water in the hair coloring base composition is at least 10 wt. %, at least 15 wt. %, at least 20 wt. %, at least 25 wt. %, at least 30 wt. %, at least 35 wt. %, or at least 40 wt. %, in each case having an optional maximum amount of up to 45, 50, 55, or 60 wt. %, based on the total weight of the hair coloring base composition.

As discussed above, the hair coloring base compositions of the instant disclosure include varying amounts of surfactants, water-soluble organic solvents, water, etc. Nonetheless, in various embodiments, the total amount (the combined amount) of surfactants, water-soluble organic solvents, and water in the hair coloring base composition is from about 60 wt. % to about 95 wt. % based on the total weight of the hair coloring base composition. In further embodiments, the total amount (the combined amount) of surfactants, water-soluble organic solvents, and water in the hair coloring base composition is from about 65 to about 95 wt. %, from about 70 to about 95 wt. %, from about 75 to about 95 wt. %, from about 80 to about 95 wt. %, from about 85 wt. % to about 95 wt. %, from about 60 to about 90 wt. %, from about 65 to about 90 wt. %, from about 70 to about 90 wt. %, from about 75 to about 90 wt. %, from about 80 to about 90 wt. %, or from about 85 to about 90 wt. %, based on the total weight of the hair coloring base composition.

In further embodiments, the total amount (the combined amount) of surfactants, water-soluble organic solvents, and water in the hair coloring base composition is at least 60 wt. %, at least 65 wt. %, at least 70 wt. %, at least 75 wt. %, at least 80 wt. %, or at least 85 wt. %, based on the total weight of the hair coloring base composition.

In various embodiments, the hair coloring composition may include one or more fatty compounds other than fatty alcohols, preferably one or more non-silicone fatty compounds. The term “non-silicone fatty compound” means a fatty compound that does not containing any silicon atoms (Si). Non-limiting examples of non-silicone fatty compounds include oils, mineral oil, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of fatty acids, fatty alcohol derivatives, and fatty acid derivatives are found in International Cosmetic Ingredient Dictionary, Sixteenth Edition, 2016, which is incorporated by reference herein in its entirety. A more exhaustive but nonlimiting list of fatty compounds is provided later, under the heading “Fatty Compounds Other Than Fatty Alcohols.”

In various embodiments, the one or more fatty compounds other than fatty alcohols are liquid fatty compounds, also referred to “oils.” “Oil” is used herein to refer to an organic compound other than a fatty alcohols that is insoluble in water at normal temperature (25° C.) and at atmospheric pressure (760 mmHg), i.e. it has a water solubility of less than 5% by weight, or less than 1% by weight, or less than 0.1% by weight. Oils have in their structure a chain of at least two siloxane groups or at least one hydrocarbon chain having at least 6 carbon atoms. Furthermore, oils are generally soluble in organic solvents in the same conditions of temperature and pressure, for example in chloroform, ethanol, benzene or decamethylcyclopentasiloxane. Furthermore, oils are liquid at ordinary temperature (25° C.) and at atmospheric pressure (760 mmHg). The oils preferably do not contain any carboxylic acid functions, i.e. they do not contain any —COON or —COO— groups. As described throughout the disclosure fatty alcohols are independent from fatty compounds and oils, i.e., even if a fatty alcohol is present in the compositions of the instant disclosure, the compositions may nonetheless be free or essentially free from fatty compounds of oils (because fatty alcohols are not included in the definition of fatty compounds and oils).

The total amount of the one or more fatty compounds in the hair coloring compositions, if present, will vary. Nonetheless, in various embodiments, the total amount of fatty compounds is from about 0.1 to about 20 wt. %, based on the total weight of the hair coloring composition. In further embodiments, the total amount of fatty compounds is from about 0.1 to about 15 wt. %, about 0.1 to about 10 wt. %, about 0.1 to about 5 wt. %, based on the total weight of the hair coloring base composition.

The total amount of the one or more oils (a type of fatty compound) in the hair coloring base compositions, if present, will vary. Nonetheless, in various embodiments, the total amount of oil is from about 0.1 to about 18 wt. %, about 0.1 to about 15 wt. %, about 0.1 to about 10 wt. %, about 0.1 to about 5 wt. %, about 0.1 to about 3 wt. %, about 0.1 to about 2 wt. %, based on the total weight of the hair coloring base composition. In further embodiments, the hair coloring compositions not more than 18 wt. % of oils, not more than 15 wt. % or oils, not more than 12 wt. % of oils, not more than 10 wt. % of oils, not more than 8 wt. % of oils, not more than 5 wt. % of oils, not more than 4 wt. % of oils, not more than 3 wt. % of oils, not more than 2 wt. % of oils, not more than 1 wt. % of oil, or may be free or essentially free from oils.

In various embodiments the hair coloring base compositions are free or essentially free from mineral oil, polybutene, hydrogenated polyisobutene, hydrogenated polydecene, polydecene, squalane, petrolatum, petroleum jelly (including liquid petroleum jelly), paraffin (including liquid paraffin), or isoparaffins, or mixtures, thereof. In further embodiments, the hair coloring base compositions are free from at least petrolatum, petroleum jelly (including liquid petroleum jelly), paraffin (including liquid paraffin), isoparaffins, and mixtures, thereof.

In various embodiments, the hair coloring base composition includes one or more cationic conditioning polymers. Nonlimiting examples include cationic polysaccharides derivatives, cationic gum derivatives, polymer derivatives of diallyldimethyl ammonium chloride, polymer derivatives of methacrylamidopropyltrimethylammonium chloride, cationic cellulose derivatives, quaternized hydroxyethyl cellulose, cationic starch derivatives, cationic guar gum derivatives (hydroxypropyl guar hydroxypropyltrimonium chloride), copolymers of acrylamide and dimethyldiallyammonium chloride, polyquaterniums, and a mixture thereof. A more exhaustive but nonlimiting list of cationic conditioning polymers is included later, under the heading “Cationic Conditioning Polymers.”

The total amount of the one or more cationic conditioning polymers, if present, will vary. Nonetheless, in various embodiments, the total amount of the one or more cationic conditioning polymers in the hair coloring base composition is from about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. %, based on the total weight of the hair coloring base composition.

In various embodiments, the hair coloring base composition includes one or more thickening agents. Nonlimiting examples of thickening agents include polyacrylate crosspolymers or crosslinked polyacrylate polymers, cationic acrylate copolymers, anionic acrylic or carboxylic acid polymers, polyacrylamide polymers, polysaccharides, gums, polyquaterniums, vinylpyrrolidone homopolymers/copolymers, C8-24 hydroxyl substituted aliphatic acid, C8-24 conjugated aliphatic acid, sugar fatty esters, polyglyceryl esters, and a mixture thereof. A more exhaustive but non-limiting list of thickening agents is included later, under the heading “Thickening Agents.”

The total amount of the one or more thickening agents, if present, will vary. For example, in various embodiments, the hair coloring base composition includes one or more thickening agents in an amount of about 0.01 to about 10 wt. %, about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, based on the total weight of the hair coloring base composition.

The hair coloring base composition may optionally include one or more conditioning agents. For example, the hair coloring base compositions may optionally include one or more glyceryl ethers as conditioning agent(s). Non-limiting examples of glyceryl ethers include glyceryl butyl ether, glyceryl isobutyl ether, glyceryl tert-butyl ether, glyceryl pentyl ether, glyceryl isopentyl ether, glyceryl hexyl ether, glyceryl isohexyl ether, glyceryl heptyl ether, glyceryl octyl ether, glyceryl ethylhexyl ether, glyceryl nonyl ether, glyceryl decyl ether, glyceryl isodecyl ether, glyceryl lauryl ether, glyceryl myristyl ether, glyceryl palmityl ether, glyceryl stearyl ether and glyceryl behenyl ether and their mixtures. Particularly useful glyceryl ethers also include glyceryl butyl ether, glyceryl isobutyl ether, glyceryl tert-butyl ether, glyceryl pentyl ether, glyceryl isopentyl ether, glyceryl hexyl ether, glyceryl isohexyl ether, glyceryl heptyl ether, glyceryl octyl ether, glyceryl ethylhexyl ether, glyceryl nonyl ether, glyceryl decyl ether, glyceryl isodecyl ether, glyceryl lauryl ether, and a mixture thereof. In some instances, glyceryl lauryl ether is particularly useful.

The total amount of the one or more conditioning agents, if present, may vary but is typically about 0.1 to about 15 wt. %, based on the total weight of the hair coloring base composition. The total amount of the one or more conditioning agents can be about 0.1 to about 10 wt. %, 0.1 to about 5 wt. %, about 0.5 to about 15 wt. %, about 0.5 to about 10 wt. %, about 0.5 to about 5 wt. %, or about 1 to about 10 wt. %, or about 1 to about 5 wt. %, based on the total weight of the hair coloring base composition.

The hair coloring compositions of the instant disclosure (hair coloring base compositions, developer compositions, and/or the ready-to-use hair coloring compositions) may optionally include (or optionally exclude) one more miscellaneous ingredients. Miscellaneous ingredients are ingredients that are compatible with the hair treatment composition but do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers, composition colorants, cationic polymers, thickening agents, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal, which is not intended to impart coloring properties to hair. Styling gels, for example, can be found in a variety of different colors (e.g., light blue, light pink, etc.) yet application of the styling gel to the hair does not change the color of the hair.

The total amount of the one or more miscellaneous ingredients, if present, will vary. Nonetheless, in various embodiments, the compositions of the instant disclosure (hair coloring base compositions, developer compositions, and/or the ready-to-use hair coloring compositions) include, if present, from about 0.001 to about 10 wt. % of one or more miscellaneous ingredients. In further embodiments, the compositions of the instant disclosure include from about 0.001 to about 5 wt. %, about 0.001 to about 3 wt. %, about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. % of one or more miscellaneous ingredients.

In various embodiments, the hair coloring base composition comprises or consists of:
(a) 2-methoxymethyl-P-phenylenediamine, and optionally one or more additional oxidative dye precursors;
(b) one or more couplers; and
(c) 3 wt. % or less, preferably 2.5 wt. % or less, more preferably 2 wt. % or less, even more preferably 1.5 wt. % or less of one or more alkalizing agents, wherein the one or more alkalizing agents is preferably monoethanolamine;
(d) about 0.1 to about 10 wt. %, preferably about 0.1 to about 5 wt. %, more preferably about 0.5 to about 3 wt. % of one or more fatty alcohols having from 12 to 24 carbon atoms, preferably chosen from cetyl alcohol, stearyl alcohol, cetearyl alcohol, behenyl alcohol, lauryl alcohol, myristic or myristyl alcohol, arachidyl alcohol, lignoceryl alcohol, oleyl alcohol, and mixtures thereof;
(e) about 5 to about 40 wt. %, preferably about 10 to about 30 wt. %, more preferably about 15 to about 30 wt. % of one or more surfactants, preferably one or more nonionic surfactants and optionally one or more anionic surfactants, in particular, wherein the one or more nonionic surfactants chosen from oxyethylenated amides, oxyethylenated fatty alcohols, and block-copolymer (polycondensate) surfactants of ethylene oxide and of propylene oxide, and a mixture thereof, more preferably chosen from oxyethylenated amides (e.g., PEG-4 rapeseedamide), oxyethylenated fatty alcohols (e.g., deceth-3), block-copolymer (polycondensate) surfactants of ethylene oxide and of propylene oxide (e.g., poloxamer 338), or a combination thereof.
(f) about 10 to about 40 wt. %, preferably about 15 to about 35 wt. %, more preferably about 15 to about 30 wt. % of one or more water-soluble organic solvents, in particular, one or more water-soluble organic solvents chosen from glycerin, mono-alcohols, polyols (polyhydric alcohols), glycols, and a mixture thereof, preferably, the one or more water-soluble solvents are chosen from glycerin, propylene glycol, butylene glycol, pentylene glycol, dipropylene glycol, hexylene glycol, ethanol, isopropanol, t-butyl alcohol, and a mixture thereof;
(g) about 25 to about 60 wt. %, preferably about 30 to about 55 wt. %, more preferably about 35 to about 50 wt. % of water;
   wherein (e), (f), and (g) are in amounts such that a combined amount of (e), (f), and (g) constitutes at least 70 wt. %, preferably at least 75 wt. %, more preferably at least 80 wt. %, and even more preferably at least 83 wt. % of the hair coloring base composition;
(h) less than 15 wt. %, preferably less than 10 wt. %, more preferably less than 5 wt. % of oils;
(i) optionally, about 0.1 to about 10 wt %, preferably about 0.1 to about 5 wt. %, more preferably about 0.1 to about 3 wt. % of one or more reducing agents, for example, chosen from potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof, preferably thioglycolic acid, thiolatic acid, salts thereof, and mixtures thereof; and
(j) optionally, about 0.01 to about 10 wt. %, preferably about 0.01 to 5 wt. %, more preferably about 0.1 to about 5 wt. % of one or more miscellaneous ingredients, for example, preservatives, fragrances, pH adjusters, salts, chelating agents, buffers, antioxidants, flavonoids, vitamins, botanical extracts, UV filtering agents, proteins, protein hydrolysates, and/or isolates, fillers, composition colorants, cationic polymers, thickening agents, and mixtures thereof;
   all percentages by weight are based on the total weight of the hair coloring base composition.

The pH of the hair coloring base composition will vary. However, the pH is typically such that when mixed with a developer composition to form a ready-to-use hair coloring composition, the ready-to-use hair coloring composition has a desired pH, e.g., a pH from about 6 to about 8, preferably about 6.1 to about 7.9, more preferably from about 6.2 to about 7.8. Developer composition are typically acidic, for example, having a pH of 2 to about 6. Therefore, to derive a ready-to-use hair coloring composition by mixing the developer composition with the hair coloring base composition, the hair coloring base composition should have a low pH, for example from about 7 to about 10, preferably from about 7.5 to about 9.5, more preferably from about 8 to less than 9.5. The pH of the hair coloring base composition, the pH of the developer composition, and the mixing ratio of hair coloring base composition to developer composition can influence the final pH of the ready-to-use hair coloring composition.

In various embodiments, the pH of the hair coloring base composition is from about 7 to about 10, about 7.5 to about 10, about 8 to about 10, from about 8.5 to about 10, from about 7 to about 9.5, from about 7.5 to about 9.5, from about 8 to about 9.5, from about 8.5 to about 9.5, from about 7 to about 9, from about 7.5 to about 9, or from about 8 to about 9.

In various embodiments, the hair coloring base compositions are free or essentially free from resorcinol and/or resorcinol derivatives. The hair color altering compositions may, in some embodiments, be free or substantially free of para-phenylenediamines (other than 2-methoxymethyl-P-phenylenediamine), resorcinol, and/or resorcinol derivatives. In some embodiments, the hair color altering compositions may be free or substantially free of one or more of mineral oil, ammonia, ammonium hydroxide, ammonium thiolactate, para-phenylenediamines (other than 2-methoxymethyl-P-phenylenediamine), resorcinol, and/or resorcinol derivatives. In other embodiments, the hair color altering compositions may be free or substantially free of mineral oil, ammonia, ammonium hydroxide, ammonium thiolactate, para-phenylenediamines (other than 2-methoxymethyl-P-phenylenediamine), resorcinol, and resorcinol derivatives.

As noted, the hair coloring base compositions of the instant disclosure are combinable with developer composition to form a ready-to-use hair coloring composition having a pH from about 6 to about 8, preferably about 6.1 to about 7.9, or more preferably about 6.2 to about 7.8. The final pH of a ready-to-use hair coloring composition will depend on the content, pH, etc., of the hair coloring base composition. In various embodiments, the hair coloring base compositions of the instant disclosure are such that they form a ready-to-use hair coloring composition having a pH from about 6 to about 8, about 6.1 to about 7.9, or about 6.2 to about 7.8, upon mixing with a developer composition comprising hydrogen peroxide in a weight ratio about 1:5 to about 5:1 (hair coloring composition:developer composition). For example, the hair coloring base compositions are such that they form a ready-to-use hair coloring composition having a pH from about 6 to about 8, about 6.1 to about 7.9, or about 6.2 to about 7.8, upon mixing with a developer composition comprising hydrogen peroxide in a weight ratio about 1:1 (hair coloring composition:developer composition), wherein the developer composition consists of:
(a) about 7.5 wt. % hydrogen peroxide;
(b) about 82 wt. % water;
(c) about 0.5 wt. % glycerin;
(d) about 2.3 wt. % of cetearyl alcohol;
(e) about 0.9 wt. % of trideceth-2 carboxamide MEA;
(f) about 0.6 wt. % ceteareth-25; and
   wherein all percentages by weight for components of the developer composition are based on the total weight of the developer composition.

In various embodiments, the instant disclosure relates to a method comprising or consisting of:
(i) obtaining the hair coloring base composition according to the instant disclosure;
(ii) obtaining a developer composition comprising:
   (a) one or more oxidizing agents; and
   (b) water;
(iii) mixing the hair coloring base composition of (i) and the developer composition of (ii) in a weight ratio of about 1:5 to about 5:1, preferably about 1:3 to about 3:1, more preferably about 1:1 to form a ready-to-use hair coloring composition having a pH of about 6 to about 8, preferably about 6.1 to about 7.9, or more preferably about 6.2 to about 7.8; and
(iv) applying the ready-to-use hair coloring composition to hair, preferably the hair of the face, more preferably beard or mustache hair;
(v) allowing the ready-to-use hair coloring composition to remain on the hair for a period of time, wherein the period of time is at least 1 minute but less than 30 minutes, preferably less than 15 minutes, more preferably less than 10 minutes, or even less than 5 minutes; and
(vi) after the period of time has lapsed, rinsing the ready-to-use hair coloring composition from the hair.

The hair coloring base compositions according to the disclosure may be mixed at or near the time of use with a developer composition (also referred to as an oxidizing composition) comprising at least one oxidizing agent. The oxidizing agent may be, for example, chosen from peroxides, persulfates, perborates, percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. Oxygen in the air may also be an oxidizing component.

In certain embodiments, the oxidizing agent is hydrogen peroxide. In various embodiments the hydrogen peroxide may be present in an aqueous solution whose titer may range from 1 to 40 volumes, such as from 5 to 40 volumes, from 5 to 30 volumes, or from 5 to 20 volumes. In certain embodiments, the oxidizing component is a 20V, 30V, or 40V hydrogen peroxide developer composition.

In other embodiments, the oxidizing agent is a persulfate and/or a monopersulfate such as, for example, potassium persulfate, sodium persulfate, ammonium persulfate, as well as mixtures thereof. In some embodiments, the oxidizing agents are chosen from hydrogen peroxide, potassium persulfate, sodium persulfate, or mixtures thereof.

The oxidizing agent may, in various embodiments, be present in the developer composition in an amount ranging from about 0.05% to about 50% by weight, such as from about 0.1% to about 30% by weight, from about 0.1% to about 20% by weight, about 1% to about 20%, about 1% to about 15%, about 1% to about 12%, about 3% to about 20%, about 3% to about 15%, about 3% to about 12%, about 5% to about 20%, about 5% to about 15%, about 5% to about 12%, about 7% to about 20%, about 7% to about 15%, about 7% to about 12%, about 9% to about 20%, about 9% to about 15%, or about 9% to about 12% by weight, based on the total weight of the developer composition.

The developer composition may contain at least one solvent, for example water, water-soluble organic solvents, or mixtures thereof. Nonlimiting examples off suitable water-soluble organic solvents for use in the developer composition, alone or in mixture with water, include but are not limited to ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, or mixtures thereof.

The developer compositions may optionally include other components typically used in developer compositions, such as, for example, thickening agents, chelants, fatty substances, ceramides, pH adjusting agents, preservatives, fragrances, surfactants, etc.

The developer composition may be in the form of a powder, gel, liquid, foam, lotion, cream, mousse, or emulsion. In certain embodiments the developer composition is aqueous and is in the form of a liquid, cream, or emulsion. In other embodiments, the developer composition is anhydrous or substantially anhydrous.

In various exemplary embodiments, the developer composition includes hydrogen peroxide. For example, hydrogen peroxide can be present in an amount of at least about 1% by weight, based on the total weight of the developer composition. In further embodiments, hydrogen peroxide is present in an amount ranging from about 0.1% to about 80% by weight, such as from about 1.0% to about 75% by weight, or from about 2% to about 10% by weight, based on the total weight of the developer composition. In further exemplary embodiments, the hydrogen peroxide may be present in the developer composition in an amount ranging from about 2% to about 25%, such as about 4% to about 20%, about 6% to about 15%, or about 7% to about 10%.

In alternative embodiments, the developer composition is substantially anhydrous. The term “substantially anhydrous” means that the developer composition is either completely free of water or contains no appreciable amount of water, for example, no more than 5% by weight, or no more than 2% by weight, or no more than 1% by weight, based on the weight of the developer composition. It should be noted that this refers, for example, to bound water, such as the water of crystallization of the salts or traces of water absorbed by the raw materials used in the preparation of the compositions according to embodiments of the disclosure.

When the developer composition is substantially anhydrous, the developer composition may comprise at least one water-soluble organic solvent. Nonlimiting examples include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

In a preferred embodiment, the developer compositions include:
one or more oxidizing agents;
one or more surfactants; and
water.

The developer compositions include one or more oxidizing agents present in an amount sufficient to develop a color when mixed with a hair coloring base composition. Non-limiting examples of oxidizing agents include hydrogen peroxide, inorganic alkali metal peroxides (such as sodium periodate and sodium peroxide), organic peroxides (such as urea peroxide and melamine peroxide), inorganic perhydrate salts (such as the alkali metal salts of perborates, percarbonates, perphosphates, persilicates, persulfates), bromates, and a mixture thereof. In some cases, the developer composition includes hydrogen peroxide.

The total amount of the one or more oxidizing agents in the developer composition may vary but is typically about 0.5 to about 50 wt. %, based on the total weight of the developer composition. In some instances, the total amount of the one or more oxidizing agents is about 0.5 to about 40 wt. %, about 1 to about 50 wt. %, about 1 to about 40 wt. %, about 1 to about 30 wt. %, about 1 to about 20 wt. %, about 1 to about 10 wt. %, about 5 to about 50 wt. %, about 5 to about 40 wt. %, about 5 to about 30 wt. %, about 5 to about 20 wt. %, or about 5 to about 10 wt. %, based on the total weight of the developer composition.

The developer composition typically includes one or more surfactants. The one or more surfactants may be anionic, amphoteric, non-ionic, zwitterionic, cationic surfactants, or a mixture thereof.

The total amount of the one or more surfactants in the developer composition can vary but is typically about 0.1 to about 25 wt. %, based on the total weight of the hair developer composition. The total amount of the one or more surfactants may be about 0.1 to about 20 wt. %, about 0.1 to about 15 wt. %, about 0.1 to about 10 wt. %, about 0.1 to about 5 wt. %, about 1 to about 25 wt. %, about 1 about 20 wt. %, about 1 to about 15 wt. %, about 1 to about 10 wt. %, or about 1 to about 5 wt. %, based on the total weight of the developer composition.

In some instances, the developer composition includes at least one or more nonionic surfactants. For example, the developer compositions may include one or more nonionic surfactants selected from the group consisting of oxyethylenated amides, fatty alcohols, oxyethylenated fatty alcohols, and mixture thereof. For example, the developer composition may include cetearyl alcohol (fatty alcohols), trideceth-2 carboxamide MEA (an oxyethylenated amide), and ceteareth-25 (an oxyethylenated fatty alcohol), or a mixture thereof.

Non-limiting examples of nonionic oxyethylenated amides are those of the following formula:

R—[(OCH2CH2)n-OCH2]ₚ—CO—N(R′)—(CH2CH2O)ₙ′H
in which:
p denotes 0 or 1,
n denotes a number ranging from 1 to 10 and preferably from 1 to 6,
n′ denotes a number ranging from 1 to 100 and preferably from 1 to 60,
R′ denotes a hydrogen atom or a CH2CH2OH radical and preferably a hydrogen atom, and
R denotes a C10-C30 and preferably C12-C22 alkyl or alkenyl radical.

Examples of these compounds include AMIDET A15 sold by the company Kao (INCI name: Trideceth-2 carboxamide MEA), ETHOMID HP 60 sold by the company Akzo Nobel (INCI name: PEG-50 Hydrogenated Palmamide) and AMIDET N sold by the company Kao (INCI name: PEG-4 Rapeseedamide). In some cases, trideceth-2 carboxamide MEA may be particularly useful.

Non-limiting examples of fatty alcohols include saturated or unsaturated and linear or branched alcohols comprising from 6 to 30 carbon atoms and preferably from 8 to 30 carbon atoms, for instance, cetyl alcohol, isostearyl alcohol, stearyl alcohol and the mixture thereof (cetylstearyl alcohol/cetearyl alcohol), octyldodecanol, 2-butyloctanol, 2-hexyldecanol, 2-undecylpentadecanol, oleyl alcohol, linolenyl alcohol, ricinoleyl alcohol, undecylenyl alcohol and linoleyl alcohol, and mixtures thereof. In some cases, cetylstearyl/cetearyl alcohol may be particularly useful.

Non-limiting examples of oxyethylenated fatty alcohols include those comprising less than 10 OE units, preferably chosen from oxyethylenated derivatives of saturated or unsaturated, linear or branched, preferably linear, C₈-C₃₀ and preferably C₁₂-C₂₂ fatty alcohols, for instance cetyl alcohol, oleyl alcohol, oleocetyl alcohol, lauryl alcohol, behenyl alcohol, cetearyl alcohol, stearyl alcohol and isostearyl alcohol, and mixtures thereof.

The oxyethylenated fatty alcohols may have an average degree of ethoxylation of 2 to 29. Non-limiting examples of oxyethylenated fatty alcohols having an average degree of ethoxylation of 2 to 29 are, for example, laureth-2, oleth-2, ceteareth-2, laneth-2, laureth-3, oleth-3, ceteareth-3, laureth-4, oleth-4, ceteareth-4, laneth-4, laureth-5, oleth-5, ceteareth-5, laneth-5, deceth-4, deceth-7, laureth-7, oleth-7, coceth-7, ceteth-7, ceteareth-7, C11-15 pareth-7, laureth-9, oleth-9, ceteareth-9, laureth-10, oleth-10, beheneth-10, ceteareth-10, laureth-12, ceteareth-12, trideceth-12, ceteth-15, laneth-15, ceteareth-15, laneth-16, ceteth-16, oleth-16, steareth-16, oleth-20, ceteth-20, ceteareth-20, laneth-20, steareth-21, ceteareth-23, ceteareth-25, ceteareth-27, and a mixture thereof.

The developer compositions may be free or essentially free of amphoteric surfactants and/or the developer compositions may be free or essentially free of cationic surfactants and/or the developer compositions may be free or essentially free of anionic surfactants.

The total amount of the one or more nonionic surfactants in the developer composition can vary but is typically about 0.1 to about 25 wt. %, based on the total weight of the hair developer composition. The total amount of the one or more nonionic surfactants may be about 0.1 to about 20 wt. %, about 0.1 to about 15 wt. %, about 0.1 to about 10 wt. %, about 0.1 to about 5 wt. %, about 1 to about 25 wt. %, about 1 about 20 wt. %, about 1 to about 15 wt. %, about 1 to about 10 wt. %, or about 1 to about 5 wt. %, based on the total weight of the developer composition.

The developer composition may include one or more water-soluble solvents. Non-limiting examples of water-soluble solvents include, for example, glycerin, alcohols (for example, C₁₋₃₀, C₁₋₁₅, C₁₋₁₀, or C₁₋₄ alcohols), organic solvents, polyols, glycols, and a mixture thereof. The water-soluble solvents useable in the developer composition include those discussed above with respect to the hair coloring base composition, which for the sake of brevity are not reiterated here.

The total amount of the one or more water-soluble solvents in the developer composition, if present, may be about 0.1 to about 25 wt. %, about 0.1 to about 20 wt. %, about 0.1 to about 15 wt. %, about 0.1 to about 10 wt. %, or about 0.1 to about 5 wt. %, based on the total weight of the developer composition.

The viscosity of the developer composition is typically about 250 to about 2000 cps at 25° C. using a #4 spindle at 100 rpm. In some cases, the viscosity of the developer composition may be about 500 to about 2500 cps, about 500 to about 2000 cps, about 500 to about 1500 cps, about 600 to about 1300 cps, or about 650 to about 1200 cps at 25° C. using a #4 spindle at 100 rpm.

In one embodiment, the developer composition includes:
about 1 to about 40 wt. %, about 1 to about 25 wt. %, or about 1 to about 15 wt. % of one or more oxidizing agents, for example, one or more oxidizing agents chosen from hydrogen peroxide, inorganic alkali metal peroxides (such as sodium periodate and sodium peroxide), organic peroxides (such as urea peroxide and melamine peroxide), inorganic perhydrate salts (such as the alkali metal salts of perborates, percarbonates, perphosphates, persilicates, persulphates), bromates, and a mixture thereof, preferably hydrogen peroxide;
about 0.1 to about 20 wt. %, about 0.1 to about 10 wt. %, or about 1 to about 10 wt. % of one or more nonionic surfactants, for example, one or more nonionic surfactants selected from the group consisting of oxyethylenated amides, fatty alcohols, oxyethylenated fatty alcohols, and a mixture thereof;
optionally, about 0.1 to about 25 wt. %, about 0.1 to about 15 wt. %, or about 0.1 to about 10 wt. % of one or more water-soluble solvents, for example, glycerin, alcohols, organic solvents, polyols, glycols, and a mixture thereof; and
about 50 to about 92 wt. %, about 60 to about 92 wt. %, or about 75 to about 90 wt. % of water;
   wherein the weight percentages are based on the total weight of the developer composition.

The hair coloring base composition and the developer composition can be mixed at a ratio of about 1:5 to about 5:1 (hair coloring base composition: developer composition). In some cases, the ratio is about 1:4 to about 4:1, about 1:3 to about 3:1, about 1:2 to about 2:1, or about 1:1 (hair coloring base composition: developer composition).

As previously noted, the hair coloring base compositions of the instant disclosure are combinable with developer composition to form a ready-to-use hair coloring composition having a pH from about 6 to about 8, preferably about 6.1 to about 7.9, or more preferably about 6.2 to about 7.8. In various embodiments, the hair coloring base compositions of the instant disclosure are such that they form a ready-to-use hair coloring composition having a pH from about 6 to about 8, about 6.1 to about 7.9, or about 6.2 to about 7.8, upon mixing with a developer composition comprising hydrogen peroxide.

In a preferred embodiment, the hair coloring base compositions of the instant disclosure provide for a pH from about 6 to about 8, preferably about 6.1 to about 7.9, or more preferably about 6.2 to about 7.8 when mixed with a developer composition in a 1:1 weight ratio, wherein the developer composition consists of:
(a) about 7.5 wt. % hydrogen peroxide;
(b) about 82 wt. % water;
(c) about 0.5 wt. % glycerin;
(d) about 2.3 wt. % of cetearyl alcohol;
(e) about 0.9 wt. % of trideceth-2 carboxamide MEA;
(f) about 0.6 wt. % ceteareth-25; and
   wherein all percentages by weight for components of the developer composition are based on the total weight of the developer composition.

A hair coloring base composition that provides for a pH from about 6 to about 8, preferably about 6.1 to about 7.9, or more preferably about 6.2 to about 7.8 when mixed with the specific developer composition described above is not required to be mixed with the specific developer composition described above during use. Rather, the specific developer composition described above can be used to assess properties of the hair coloring base composition. In other words, the specific developer composition described above can be used in a method for determining whether a particular hair coloring base composition is capable of forming a ready-to-use hair coloring composition according to the instant disclosure.

The instant disclosure relates to ready-to-use hair coloring composition comprising a combination of a hair coloring base composition according to the instant disclosure an a developer composition comprising an oxidizing agent. In various embodiments, the developer composition is chosen from the developer compositions described throughout the instant disclosure. Nonlimiting examples of oxidizing agent useful in developer compositions include hydrogen peroxide, inorganic alkali metal peroxides (such as sodium periodate and sodium peroxide), organic peroxides (such as urea peroxide and melamine peroxide), inorganic perhydrate salts (such as the alkali metal salts of perborates, percarbonates, perphosphates, persilicates, persulphates), bromates, and a mixture thereof. I certain embodiments, the oxidizing agent is a peroxo compound, preferably hydrogen peroxide. In various embodiments, the ready-to-use hair coloring composition comprises or consists of a hair coloring base composition and a developer composition combined in a weight ratio of about 5:1 to about 1:5, about 4:1 to about 1:4, about 3:1 to about 1:3, about 1:2 to about 2:1, or about 1:1.

In various embodiments, the ready-to-use hair coloring composition comprises or consists of a combination of hair coloring base composition and a developer composition combined in a weight ratio of about 5:1 to about 1:5, about 4:1 to about 1:4, about 3:1 to about 1:3, about 1:2 to about 2:1, or about 1:1; wherein

(I) the hair coloring base composition comprises or consists of:
(a) 2-methoxymethyl-P-phenylenediamine, and optionally one or more additional oxidative dye precursors;
(b) one or more couplers;
(c) less than 3 wt. %, preferably less than 2.5 wt. %, more preferably less than 2 wt. %, even more preferably less than 1.5 wt. % of one or more organic and/or mineral alkalizing agents, based on the total weight of the hair coloring base composition;
(d) one or more fatty alcohols;
(e) one more surfactants;
(f) optionally one or more water-soluble organic solvents;
(g) water.
   wherein (e), (f), and (g) are in amounts such that a combined amount of (e), (f), and (g) constitutes at least 70 wt. %, preferably at least 75 wt. %, more preferably at least 80 wt. % of the hair coloring base composition; and
   all percentages by weight are based on the total weight of the hair coloring base composition; and
      (II) the developer composition comprises or consists of:
(a) one or more oxidizing agents, for example, one or more oxidizing agents chosen from hydrogen peroxide, inorganic alkali metal peroxides (such as sodium periodate and sodium peroxide), organic peroxides (such as urea peroxide and melamine peroxide), inorganic perhydrate salts (such as the alkali metal salts of perborates, percarbonates, perphosphates, persilicates, persulphates), bromates, and a mixture thereof, preferably hydrogen peroxide;
(b) one or more nonionic surfactants, for example, one or more nonionic surfactants selected from the group consisting of oxyethylenated amides, fatty alcohols, oxyethylenated fatty alcohols, and a mixture thereof;
(c) optionally, one or more water-soluble solvents, for example, glycerin, alcohols, organic solvents, polyols, glycols, and a mixture thereof; and
(d) about 50 to about 92 wt. %, about 60 to about 92 wt. %, or about 75 to about 90 wt. % of water;
   wherein the weight percentages are based on the total weight of the developer composition; and
   wherein the ready-to-use hair coloring composition:
   (i) has a pH from about 6 to about 8, preferably about 6.1 to about 7.9, more preferably from about 6.2 to about 7.8;
   (ii) comprises 2 wt. % or less, preferably 1.5 wt. % or less, more preferably 1 wt. % or less, even more preferably 0.7 wt. % or less of one or more alkalizing agents, wherein the one or more alkalizing agents is preferably monoethanolamine; and
   (iii) is optionally, free or essentially free from ammonia, ammonium hydroxide, and ammonium ions.

In further embodiments, the ready-to-use hair coloring composition comprises or consists of a combination of hair coloring base composition and a developer composition combined in a weight ratio of about 5:1 to about 1:5, about 4:1 to about 1:4, about 3:1 to about 1:3, about 1:2 to about 2:1, or about 1:1; wherein

(I) the hair coloring base composition comprises or consists of:
(a) 2-methoxymethyl-P-phenylenediamine, and optionally one or more additional oxidative dye precursors;
(b) one or more couplers; and
(c) 3 wt. % or less, preferably 2.5 wt. % or less, more preferably 2 wt. % or less, even more preferably 1.5 wt. % or less of one or more alkalizing agents, wherein the one or more alkalizing agents is preferably monoethanolamine;
(d) about 0.1 to about 10 wt. %, preferably about 0.1 to about 5 wt. %, more preferably about 0.5 to about 3 wt. % of one or more fatty alcohols having from 12 to 24 carbon atoms, preferably chosen from cetyl alcohol, stearyl alcohol, cetearyl alcohol, behenyl alcohol, lauryl alcohol, myristic or myristyl alcohol, arachidyl alcohol, lignoceryl alcohol, oleyl alcohol, and mixtures thereof;
(e) about 5 to about 40 wt. %, preferably about 10 to about 30 wt. %, more preferably about 15 to about 30 wt. % of one or more surfactants, preferably one or more nonionic surfactants and optionally one or more anionic surfactants, in particular, wherein the one or more nonionic surfactants chosen from oxyethylenated amides, oxyethylenated fatty alcohols, and block-copolymer (polycondensate) surfactants of ethylene oxide and of propylene oxide, and a mixture thereof, more preferably chosen from oxyethylenated amides (e.g., PEG-4 rapeseedamide), oxyethylenated fatty alcohols (e.g., deceth-3), block-copolymer (polycondensate) surfactants of ethylene oxide and of propylene oxide (e.g., poloxamer 338), or a combination thereof.
(f) about 10 to about 40 wt. %, preferably about 15 to about 35 wt. %, more preferably about 15 to about 30 wt. % of one or more water-soluble organic solvents, in particular, one or more water-soluble organic solvents chosen from glycerin, mono-alcohols, polyols (polyhydric alcohols), glycols, and a mixture thereof, preferably, the one or more water-soluble solvents are chosen from glycerin, propylene glycol, butylene glycol, pentylene glycol, dipropylene glycol, hexylene glycol, ethanol, isopropanol, t-butyl alcohol, and a mixture thereof;
(g) about 25 to about 60 wt. %, preferably about 30 to about 55 wt. %, more preferably about 35 to about 50 wt. % of water;
   wherein (e), (f), and (g) are in amounts such that a combined amount of (e), (f), and (g) constitutes at least 70 wt. %, preferably at least 75 wt. %, more preferably at least 80 wt. %, and even more preferably at least 83 wt. % of the hair coloring base composition;
(h) less than 15 wt. %, preferably less than 10 wt. %, more preferably less than 5 wt. % of oils;
(i) optionally, about 0.1 to about 10 wt %, preferably about 0.1 to about 5 wt. %, more preferably about 0.1 to about 3 wt. % of one or more reducing agents, for example, chosen from potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof, preferably thioglycolic acid, thiolatic acid, salts thereof, and mixtures thereof; and
(j) optionally, about 0.01 to about 10 wt. %, preferably about 0.01 to 5 wt. %, more preferably about 0.1 to about 5 wt. % of one or more miscellaneous ingredients, for example, preservatives, fragrances, pH adjusters, salts, chelating agents, buffers, antioxidants, flavonoids, vitamins, botanical extracts, UV filtering agents, proteins, protein hydrolysates, and/or isolates, fillers, composition colorants, cationic polymers, thickening agents, and mixtures thereof;
   wherein percentages by weight are based on the total weight of the hair coloring base composition; and
      (II) the developer composition comprises or consists of:
(a) about 1 to about 40 wt. %, about 1 to about 25 wt. %, or about 1 to about 15 wt. % of one or more oxidizing agents, for example, one or more oxidizing agents chosen from hydrogen peroxide, inorganic alkali metal peroxides (such as sodium periodate and sodium peroxide), organic peroxides (such as urea peroxide and melamine peroxide), inorganic perhydrate salts (such as the alkali metal salts of perborates, percarbonates, perphosphates, persilicates, persulphates), bromates, and a mixture thereof, preferably hydrogen peroxide;
(b) about 0.1 to about 20 wt. %, about 0.1 to about 10 wt. %, or about 1 to about 10 wt. % of one or more nonionic surfactants, for example, one or more nonionic surfactants selected from the group consisting of oxyethylenated amides, fatty alcohols, oxyethylenated fatty alcohols, and a mixture thereof;
(c) optionally, about 0.1 to about 25 wt. %, about 0.1 to about 15 wt. %, or about 0.1 to about 10 wt. % of one or more water-soluble solvents, for example, glycerin, alcohols, organic solvents, polyols, glycols, and a mixture thereof; and
(d) about 50 to about 92 wt. %, about 60 to about 92 wt. %, or about 75 to about 90 wt. % of water;
   wherein the weight percentages are based on the total weight of the developer composition; and
   wherein the ready-to-use hair coloring composition:
   (i) has a pH from about 6 to about 8, preferably about 6.1 to about 7.9, more preferably from about 6.2 to about 7.8;
   (ii) comprises 2 wt. % or less, preferably 1.5 wt. % or less, more preferably 1 wt. % or less, even more preferably 0.7 wt. % or less of one or more alkalizing agents, wherein the one or more alkalizing agents is preferably monoethanolamine; and
   (iii) is optionally, free or essentially free from ammonia, ammonium hydroxide, and ammonium ions.

### Oxidation Dye Precursors

The oxidation dyes are generally chosen from one or more oxidation bases optionally combined with one or more couplers. By way of example, the oxidation bases are chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N, N-bis(.beta.-hydroxyethyl)-para-phenylenediamine, 4-N,N-bis(.beta.-hydroxyethyl)amino-2-methylaniline, 4-N,N-bis(.beta.-hydroxyethyl)amino-2-chloroaniline, 2-.beta.-hydroxyethyl-para-phenylenediamine, 2-methoxymethyl-para-phenylenediamine, 2-fluoro-para-phenylenediamine, 2-isopropyl-para-phenylenediamine, N-(.beta.-hydroxypropyl)-para-phenylenediamine, 2-hydroxymethyl-para-phenylenediamine, N,N-dimethyl-3-methyl-para-phenylenediamine, N-ethyl-N-(.beta.-hydroxyethyl)-para-phenylenediamine, N-(.beta.,.gamma.-dihydroxypropyl)-para-phenylenediamine, N-(4′-aminophenyl)-para-phenylenediamine, N-phenyl-para-phenylenediamine, 2-.beta.-hydroxyethyloxy-para-phenylenediamine, 2-.beta.-acetylaminoethyloxy-para-phenylenediamine, N-(.beta.-methoxyethyl)-para-phenylenediamine, 4-aminophenylpyrrolidine, 2-thienyl-para-phenylenediamine, 2-.beta.-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-.beta.-hydroxyethyl-para-phenylenediamine, 2-.beta.-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(.beta.-hydroxyethyl)-para-phenylenediamine, 2-chloro-para-phenylenediamine and 2-.beta.-acetylaminoethyloxy-para-phenylenediamine, and the addition salts thereof with an acid, are particularly preferred.

Among the bis(phenyl)alkylenediamines that may be mentioned, for example, are N,N′-bis(beta.-hydroxyethyl)-N,N′-bis(4′-aminophenyl)-1,3-diaminopropano-I, N,N′-bis(.beta.-hydroxyethyl)-N,N′-bis(4′-aminophenyl)ethylenediamine, N,N′-bis(4-aminophenyl)tetramethylenediamine, N,N′-bis(.beta.-hydroxyethyl)-N,N′-bis(4-aminophenyl)tetramethylenediamin-e, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(.beta.-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds such as 2,5-diaminopyridine, 2-(4-methoxyphenyl)amino-3-aminopyridine and 3,4-diaminopyridine, and the addition salts thereof. Other pyridine oxidation bases that are useful in the present disclosure are the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-.quadrature.-hydroxyethoxy-3-amino-pyrazolo[1,5-a]pyridine; 2-(4-dimethylpyperazinium-1-yl)-3-amino-pyrazolo[1,5-a]pyridine; and the addition salts thereof.

More particularly oxidation bases that are useful in the present disclosure are selected from 3-aminopyrazolo-[1,5-a]-pyridines and preferably substituted on carbon atom 2 by:
(a) one (di)(C₁-C₆)(alkyl)amino group wherein said alkyl group can be substituted by at least one hydroxy, amino, imidazolium group;
(b) one heterocycloalkyl group containing from 5 to 7 members chain, and from 1 to 3 heteroatoms, potentially cationic, potentially substituted by one or more (C₁-C₆-alkyl, such as di(C1-C4)alkylpiperazinium; or
(c) one (C₁-C₆)alkoxy potentially substituted by one or more hydroxy groups such as .quadrature.-hydroxyalkoxy, and the addition salts thereof.

Among the pyrimidine derivatives that may be mentioned are compounds such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are compounds such as 4,5-diamino-1-methyl-pyrazole, 4,5-diamino-1-(.beta.-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-14.beta.-hydroxyethyl)-3-methylpyrazole, 4,5-diamino-1-ethyl-3-methyl-pyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(.beta.-hydroxyethyl)amino-1-methylpyrazole, and the addition salts thereof. 4,5-Diamino-1-(.beta.-methoxyethyl)pyrazole may also be used.

Pyrazole derivatives that may also be mentioned include diamino-N,N-dihydropyrazolopyrazolones such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-on-e, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-β-hydroxy-6,7-dihydro-1H,5H-pyrazolo[1,2-a]pyrazol-1-o-ne. 2,3-Diamino-6,7-dihydro-1H,5H-pyrazolo[1,2-a]pyrazol-1-one and/or a salt thereof will preferably be used. 4,5-Diamino-1-(.beta.-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

In various embodiments, the hair coloring base composition is free or essentially free from resorcinol and/or resorcinol derivatives. Nonlimiting examples of resorcinol derivates that may be excluded from the hair coloring base compositions include 3,5-hydroxybenzyl, 3,5-dihydroxybenzylamine, 3,5-dihydroxyphenlacetic acid, 2,4,5,6-tetraaminopyrimidine, p-phenylenediamine, p-toluylenediamine, 2,5-diaminoanisole, 2-chloro-o-phenylenediamine, N-ethyl-N-(2-hydroxyethyl)-p-phenylenediamine, N-butyl-N-sulfobutyl-p-phenylenediamine, N,N-bis-(2-hydroxyethyl)-p-phenylenediamine, N-methyl-p-phenylenediamine, N-(2-methoxyethyl)-p-phenylenediamine, N-(2-hydroxypropyl)-p-phenylenediamine, p-aminophenol, and combinations thereof.

### Couplers

The couplers include those conventionally used in oxidative methods of coloring hair, for example, meta-aminophenols, meta-phenylenediamines and meta-diphenols, naphthols, mono- or polyhydroxylated naphthalene derivatives, and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, benzomorpholine derivatives, pyrazoloazole derivatives, pyrroloazole derivatives, imidazoloazole derivatives, pyrazolopyrimidine derivatives, pyrazoline-3,5-dione derivatives, pyrrolo[3,2-d]oxazole derivatives, pyrazolo[3,4-d]thiazole derivatives, thiazoloazole S-oxide derivatives, thiazoloazole S,S-dioxide derivatives, and the acid addition salts thereof.

Suitable color couplers include, for example, those having the following general formula:

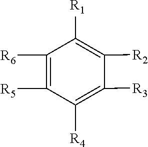

wherein R₁ is unsubstituted hydroxy or amino, or hydroxy or amino substituted with one or more C₁₋₆ hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁_₆ alkoxy, C₁₋₆ hydroxyalkyl, or C₁₋₆ alkyl, or R₃ and R₄ together may form a methylenedioxy or ethylenedioxy group. Examples of such compounds include meta-derivatives such as phenols, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methyl pyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, β-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1, 3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, β-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1, 3-dihydroxybenzene, 1-hydroxy-3-amino-benzene, 1-hydroxy-3-(carbamoylmethylamino)benzene, β-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, β-hydroxyindole, β-hydroxyindoline, 2,4-diamionphenoxyethanol, and mixtures thereof.

Other couplers may be chosen, for example, from 2,4-diamino-1-(β-hydroxyethyloxy)benzene, 2-methyl-5-aminophenol, 5-N-(β-hydroxyethyl) amino-2-methylphenol, 3-aminophenol, 1,3-dihydroxybenzene, 1,3-dihydroxy-2-methylbenzene, 4-chloro-1,3-dihydroxybenzene, 2,4-diamino 1-(β-hydroxyethyloxy) benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, β-hydroxyindole, 4-hydroxyindole, 4-hydroxy-N-methylindole, β-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2, 6-dimethylpyrazolo[1,5-b]-1,2,4-triazole, 6-methyl pyrazolo[1,5-a]-benzimidazole, and the acid addition salts thereof.

In one embodiment, the couplers include resorcinol, 1-naphthol, 2-methylresorcinol, 4-amino-2-hydroxy toluene, m-aminophenol, 2,4-diaminophenoxyethanol, phenyl methyl pyrazolone, hydroxybenzomorpholine, 2-methyl-5-hydroxyetyylaminophenol, 6-hydroxyindole, 2-amino-3-hydroxypyridine, 5-amino-6-chloro-o-cresol, 4-chlororesorcinol, their salts, and mixtures thereof.

In general, the acid addition salts of the oxidation bases and couplers may be chosen from hydrochlorides, hydrobromides, sulfates, tartrates, lactates, and acetates.

### Nonionic Surfactants

Nonionic surfactants are compounds well known in themselves (see, e.g., in this regard, “Handbook of Surfactants” by M. R. Porter, Blackie & Son publishers (Glasgow and London), 1991, pp. 116-178), which is incorporated herein by reference in its entirety.

The nonionic surfactant can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N-(C₆-C₂₄)alkylglucamine derivatives, amine oxides such as (C₁₀-C₁₄)alkylamine oxides or N-(C₁₀-C₁₄)acylaminopropylmorpholine oxides; and a mixture thereof.

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Examples of oxyalkylenated nonionic surfactants that may be mentioned include: oxyalkylenated (C₈-C₂₄)alkylphenols, saturated or unsaturated, linear or branched, oxyalkylenated C₈-C₃₀ alcohols, saturated or unsaturated, linear or branched, oxyalkylenated C₈-C₃₀ amides, esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol, saturated or unsaturated, oxyalkylenated plant oils, condensates of ethylene oxide and/or of propylene oxide, inter alia, alone or as mixtures.

The surfactants preferably contain a number of moles of ethylene oxide and/or of propylene oxide of between 2 and 100 and most preferably between 2 and 50.

In accordance with one preferred embodiment of the invention, the oxyalkylenated nonionic surfactants are chosen from oxyethylenated C₈-C₃₀ alcohols.

Examples of ethoxylated fatty alcohols (or C₈-C₃₀ alcohols) that may be mentioned include the adducts of ethylene oxide with lauryl alcohol, especially those containing from 9 to 50 oxyethylene groups and more particularly those containing from 10 to 25 oxyethylene groups (Laureth-10 to Laureth-25); the adducts of ethylene oxide with behenyl alcohol, especially those containing from 9 to 50 oxyethylene groups (Beheneth-9 to Beheneth-50); the adducts of ethylene oxide with cetearyl alcohol (mixture of cetyl alcohol and stearyl alcohol), especially those containing from 10 to 30 oxyethylene groups (Ceteareth-10 to Ceteareth-30); the adducts of ethylene oxide with cetyl alcohol, especially those containing from 10 to 30 oxyethylene groups (Ceteth-10 to Ceteth-30); the adducts of ethylene oxide with stearyl alcohol, especially those containing from 10 to 30 oxyethylene groups (Steareth-10 to Steareth-30); the adducts of ethylene oxide with isostearyl alcohol, especially those containing from 10 to 50 oxyethylene groups (Isosteareth-10 to Isosteareth-50); and a mixture thereof.

As examples of polyglycerolated nonionic surfactants, polyglycerolated C₈-C₄₀ alcohols are preferably used.

In particular, the polyglycerolated C₈-C₄₀ alcohols correspond to the following formula:

RO—[CH₂—CH(CH₂OH)—O]ₘ—H or RO—[CH(CH₂OH)—CH₂O]ₘ—H
in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and a mixture thereof.

Examples of ethoxylated fatty esters that may be mentioned include the adducts of ethylene oxide with esters of lauric acid, palmitic acid, stearic acid or behenic acid, and a mixture thereof, especially those containing from 9 to 100 oxyethylene groups, such as PEG-9 to PEG-50 laurate; PEG-9 to PEG-50 palmitate; PEG-9 to PEG-50 stearate; PEG-9 to PEG-50 palmitostearate; PEG-9 to PEG-50 behenate; polyethylene glycol 100 EO monostearate; and a mixture thereof.

As glyceryl esters of fatty acids, glyceryl stearate (glyceryl mono-, di- and/or tristearate) (glyceryl stearate) or glyceryl ricinoleate and a mixture thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Croda, and a product containing glyceryl stearate (glyceryl mono- and distearate) and potassium stearate, can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Croda.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (Methyl glucose dioleate), the mixed ester of methylglucoside and the mixture oleic acid/hydroxystearic acid (Methyl glucose dioleate/hydroxystearate), the ester of methylglucoside and isostearic acid (Methyl glucose isostearate), the ester of methylglucoside and lauric acid (Methyl glucose laurate), the mixture of monoester and diester of methylglucoside and isostearic acid (Methyl glucose sesqui-isostearate), the mixture of monoester and diester of methylglucoside and stearic acid (Methyl glucose sesquistearate) and in particular the product marketed under the name Glucate SS by Lubrizol, and a mixture thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (PEG-20 methyl glucose distearate) such as the product marketed under the name GLUCAM E-20 DISTEARATE by Lubrizol, the polyethylene glycol ether of the mixture of monoester and diester of methyl-glucose and stearic acid with about 20 moles of ethylene oxide (PEG-20 methyl glucose sesquistearate) and in particular the product marketed under the name GLUCAMATE SSE-20 by Lubrizol, and a mixture thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLATAREN 2000 by BASF, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by BASF, coco-glucoside such as the product marketed under the name PLANTACARE 818/UP by BASF, cetostearyl glucoside possibly mixed with cetostearyl alcohol, marketed for example under the name MONTANOV 68 by Seppic, under the name TEGO-CARE CG90 by Evonik, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and a mixture thereof can in particular be cited.

Mixtures of glycerides of alkoxylated plant oils such as mixtures of ethoxylated (200 EO) palm and copra (7 EO) glycerides can also be cited.

The nonionic surfactant may be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

Mixtures of these oxyethylenated derivatives of fatty alcohols and of fatty esters may also be used.

In some case, the nonionic surfactant is a fatty alkanolamide. Non-limiting examples of fatty alkanolamides that may be used include cocamide MEA, cocamide DEA, soyamide DEA, lauramide DEA, oleamide MIPA, stearamide MEA, myristamide DEA, stearamide DEA, oleylamide DEA, tallowamide DEA lauramide MIPA, tallowamide MEA, isostearamide DEA, isostearamide MEA, and a mixture thereof.

In some cases, the nonionic surfactant is an oxyethylenated amide such as PEG-4 rapeseedamide.

### Fatty Compounds Other Than Fatty Alcohols

Fatty acids useful herein include those having from about 10 to about 30 carbon atoms, from about 12 to about 22 carbon atoms, and from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of polyglycerol esters of fatty acids include those of the following formula:

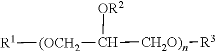

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. For example, nonionic polyglycerol esters of fatty acids include polyglyceryl-5 laurate,

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substitued fatty acids, and a mixture thereof. Nonlimiting examples of fatty acid derivatives inlcude ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a metling point of 40° C. or higher, 45° C. or higher, 50° C. or higher. The high melting point fatty compound may be selected from the group consisting of fatty acids, fatty alcohol derivatives, fatty acid derivatives, and mixtures thereof. Nonlimiting examples of the high melting point compounds are found in the International Cosmetic Ingredient Dictionary, Sixteenth Edition, 2016, which is incorporated by reference herein in its entirety.

In some instances, fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

Mention may be made, among the waxes capable of being used as non-silicone fatty compounds, of animal waxes, such as beeswax; vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

In some instance, the fatty compounds include one or more non-silicone oils. The oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

### Cationic Conditioning Polymers

Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

In certain embodiments, the one or more cationic conditioning polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride,

Non-limiting examples of cationic starch include starch hHydroxypropyltrimonium chloride, hydroxypropyl oxidized starch PG trimonium chloride, and a mixture thereof.

In various embodiments, the one or more cationic conditioning polymers are chosen from polyquaterniums. Nonlimiting examples include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), Polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), Polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quatemized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), Polyquaternium-47 (terpolymer of acrylic acid, methacrylamidopropyl trimethylammonium chloride, and methyl acrylate), and/or Polyquaternium-67.

In various embodiments, the one or more cationic conditioning polymers are chosen from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

The cationic polymers may be a polyquaternium. In certain embodiments, the cationic surfactants may be polyquaterniums selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In some embodiments, the one or more cationic conditioning polymers are chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The cationic conditioning polymers may be homopolymers or formed from two or more types of monomers. The molecular weight of the polymer may be between 5,000 and 10,000,000, typically at least 10,000, and preferably in the range 100,000 to about 2,000,000. These polymers will typically have cationic nitrogen containing groups such as quaternary ammonium or protonated amino groups, or a mixture thereof.

The cationic charge density is suitably at least 0.1 meq/g, preferably above 0.8 or higher. In some instances, the cationic charge density does not exceed 3 meq/g, or does not exceed 2 meq/g. The charge density can be measured using the Kjeldahl method and can be within the above limits at the desired pH of use, which will in general be from about 3 to 9 and preferably between 4 and 8.

The cationic nitrogen-containing group will generally be present as a substituent on a fraction of the total monomer units of the cationic conditioning polymer. Thus when the polymer is not a homopolymer it can contain spacer non-cationic monomer units.

Suitable cationic conditioning polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C₁-C₇ alkyl groups, more preferably C₁-C₃ alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition.

Amine substituted vinyl monomers and amines can be polymerized in the amine form and then converted to ammonium by quaternization.

Suitable cationic amino and quaternary ammonium monomers include, for example, vinyl compounds substituted with dialkyl amincalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these monomers are preferably lower alkyls such as the C₁-C₃ alkyls, more preferably C₁ and C₂ alkyls.

Suitable amine-substituted vinyl monomers include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic conditioning polymers can comprise mixtures of monomer units derived from amine- and/or quaternary ammonium-substituted monomer and/or compatible spacer monomers.

Suitable cationic conditioning polymers include, for example: copolymers of 1-vinyl-2-pyrrolidine and 1-vinyl-3-methyl-imidazolium salt (e.g., Chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic conditioning polymers that can be used include polysaccharide polymers, such as cationic cellulose derivatives and cationic starch derivatives. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). These materials are available from Amerchol Corp. (Edison, N.J., USA) under the tradename Polymer LM-200.

Other cationic conditioning polymers that can be used include cationic guar gum derivatives, such as guar hydroxypropyltrimonium chloride.

Polyquaterniums include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quatemized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), and Polyquaternium-47 (terpolymer of acrylic acid, methacrylamidopropyl trimethylammonium chloride, and methyl acrylate).

### Thickening Agents

Thickening agents that may be mentioned include the following:

a. Carboxylic acid or carboxylate based homopolymer or co-polymer, which can be linear or crosslinked: These polymers contain one or more monomers derived from acrylic acid, substituted acrylic acids, and salts and esters of these acrylic acids (acrylates) and the substituted acrylic acids. Commercially available polymers include those sold under the trade names CARBOPOL, ACRYSOL, POLYGEL, SOKALAN, CARBOPOL ULTREZ, and POLYGEL. Examples of commercially available carboxylic acid polymers include the carbomers, which are homopolymers of acrylic acid crosslinked with allyl ethers of sucrose or pentaerytritol. The carbomers are available as the CARBOPOL 900 series from B.F. Goodrich (e.g., CARBOPOL 954). In addition, other suitable carboxylic acid polymeric agents include ULTREZ 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL 1342, CARBOPOL 1382, PEMULEN TR-1, and PEMULEN TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is aviable in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw maerial known under the tradename of SIMULQUAT HC 305 from Seppic.

In certain embodiments, the carboxylic acid or carboxylate polymer thickeners useful herein are those selected from carbomers, acrylates/C10-C30 alkyl acrylate crosspolymers, polyacrylate crosspolymer-6, acrylamidopropyltrimonium chloride/acrylates copolymer, and mixtures thereof.

b. Celluloses: Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some instances, the cellulose is selected from water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is preferably hydroxypropylcellulose (HPC).

c. Polyvinylpyrrolidone (PVP) and co-polymers: Non-limiting examples include Polyvinylpyrrolidone(PVP), Polyvinylpyrrolidone(PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes LUVISKOL K30, K85, K90 available from BASF. Commerically available copolymers of vinylpyrrolidone and vinylacetate include LUVISKOL VA37, VA64 available from BASF; copolymers of vinylpyrrolidone, methacrylamide, and vinylimidazole (INCI: VP/Methacrylamide/Vinyl Imidazole Copolymer) is commercially available as LUVISET from BASF. In some instances, PVP and PVP/VA copolymer are preferred.

d. Sucrose esters: Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.

e. Polyglyceryl esters: Non-limiting polyglycerol esters of fatty acids (polygylceryl esters) include those of the following formula:

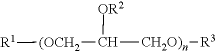

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

f. C8-24 hydroxyl substituted aliphatic acid and C8-24 conjugated aliphatic acid: Non-limiting examples include conjugated linoleic acid, cis-parinaric acid, trans-7-octadecenoic acid, cis-5,8,11,14,17-eicosapentanoic acid, cis-4,7,10,13,16,19-docosahexenoic acid, columbinic acid, linolenelaidic acid, ricinolaidic acid, stearidonic acid, 2-hydroxystearic acid, alpha-linolenic acid, arachidonic acid, cis-11,14-eicosadienoic acid, linolelaidic acid, monopetroselinic acid, petroselinic acid, ricinoleic acid, trans-vaccenic acid, cis-11,14,17-eicosatrienoic acid, cis-5-eicosenoic acid, cis-8,11,14-eicosatrienoic acid, hexadecatrienoic acid, palmitoleic acid, petroselaidic acid, trans trans farnesol, cis-13,16-docosadienoic acid, cis-vaccenic acid, cis-11-eicosenoic acid, cis-13,16,19-docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, pinolenic acid, trans-13-octadecenoic acid. More preferably, the aliphatic acid comprises 12-hydroxystearic acid, conjugated linoleic acid, or a mixture thereof.

g. Gums: Non-limiting examples of gums include gum arabic, tragacanth gum, karaya gum, guar gum, gellan gum, tara gum, locust bean gum, tamarind gum, xanthan gum, locust bean gum, Seneca gum, sclerotium gum, gellan gum, etc.

### Viscosity

The viscosity of the hair coloring base composition is typically about 25 to about 150 cps at 25° C. using a #1 spindle at 100 rpm. By way of example only, the viscosity of the hair coloring base compositions may range from about 20 cps to about 150 cps, such as, for example, from about 20 to about 125 cps, about 20 to about 100 cps, about 20 to about 85 cps, about 20 to about 70 cps, about 25 to about 125 cps, about 25 to about 100 cps, about 25 to about 85 cps, about 25 to about 70 cps, about 30 to about 125 cps, about 30 to about 100 cps, about 30 to about 85 cps, about 30 to about 70 cps, about 50 to about 100 cps, about 60 to about 100 cps, about 70 to about 100 cps, about 50 to about 90 cps, about 50 to about 80 cps, or about 50 to about 75 cps, when measured at 25° C. using a M3 spindle at 100 rpm, for example, using a Rheomat RM 180.

In some non-limiting embodiments where the developer composition is liquid, e.g. aqueous, the developer composition may have a viscosity ranging from about 250 to about 2000 cps, such as, for example, from about 500 to about 2500 cps, about 500 to about 2000 cps, about 500 to about 1500 cps, about 600 to about 1300 cps, or about 650 to about 1200 cps when measured at 25° C. using a #4 spindle at 100 rpm, for example, using a Rheomat RM 180.

By way of example, the viscosity of the ready-to-use hair coloring composition can range from about 250 cps to about 2500 cps, such as, for example, from about 250 to about 2000 cps, about 250 to about 1800 cps, about 250 to about 1600 cps, about 300 to about 2000 cps, about 300 to about 1800 cps, about 300 to about 1600 cps, about 350 to about 2000 cps, about 350 to about 1800 cps, about 350 to about 1600 cps, about 400 to about 2000 cps, about 400 to about 1800 cps, about 400 to about 1600 cps, about 450 to about 2000 cps, about 450 to about 1800 cps, about 450 to about 1600 cps, about 500 to about 2000 cps, about 500 to about 1800 cps, or about 500 to about 1600 cps, when measured at 25° C. using a M3 spindle at 100 rpm, for example, using a Rheomat RM 180.

### Methods

The compositions of the instant disclosure are useful in methods for making ready-to-use hair coloring compositions and for methods of coloring hair. Methods for making a ready-to-use hair coloring composition include combining one or more hair coloring base compositions according to the instant disclosure with one or more developer compositions, including one or more developer compositions of the instant disclosure. However, the methods are not limited to developer compositions of the instant disclosure. In various embodiments, a coloring base composition is combined with a developer composition to form a ready-to-use hair coloring composition having a pH of about 6 to about 8, preferably about 6.1 to about 7.9, or more preferably about 6.2 to about 7.8. The one or more hair coloring base composition can be combined with the one or more developer compositions in a weight ratio of about 1:5 to about 5:1 (hair coloring base composition: developer composition). In some cases, the weight ratio is about 1:4 to about 4:1, about 1:3 to about 3:1, about 1:2 to about 2:1, or about 1:1 (hair coloring base composition: developer composition), provided that the pH of the ready-to-use hair coloring composition has the desired pH.

Methods for coloring hair, especially facial hair (beard and/or mustache) include applying a ready-to-use hair coloring composition of the instant disclosure to the hair. The ready-to-use hair coloring composition may be prepared as described above, i.e., by combining one or more hair coloring base compositions according to the instant disclosure with one or more developer compositions. The ready-to-use hair coloring composition is typically used (applied to hair) shortly after formation, i.e. shortly after the hair coloring base composition is combined with the developer composition to form a ready-to-use hair coloring composition, the ready-to-use hair coloring composition is applied to hair. In various embodiments, the ready-to-use hair coloring composition is applied to hair within about 1 hour after formation. In further embodiments, the ready-to-use hair coloring composition is applied to the immediately after formation up to about 1 hour after formation, up to about 45 minutes after formation, up to about 30 minutes after formation, or up to about 15 minutes after formation.

The ready-to-use hair coloring composition may be applied to hair of the head, hair of the body, facial hair (including beard hair and mustache hair), eyelashes, and/or eyebrows. In certain embodiments, the ready-to-use hair coloring composition is applied to facial hair, preferably beard hair and/or mustache hair. In certain embodiments, the hair to be treated (colored) has been previously chemically treated, for example, previously bleached, previously colored, previously permed, previously straightened, etc. In further embodiments, the hair to be treated (colored) includes naturally grey hair, i.e., hair which has developed a loss of natural pigmentation and/or melanin.

After application to hair, the ready-to-use hair coloring composition is allowed to remain on the hair for a period of time, for example, a period of time sufficient to achieve a desired change in the color of the hair. For example, the ready-to-use hair coloring composition may be allowed to remain on the hair for up to 1 hour, such as from about 1 minute to about 45 minutes, about 1 minute to about 30 minutes, about 1 minute to about 15 minutes, about 2 minutes to about 1 hour, about 2 minutes to about 45 minutes, about 2 minutes to about 30 minutes, about 2 minutes to about 15 minutes, about 2 minutes to about 10 minutes, about 3 minutes to about 30 minutes, about 3 minutes to about 25 minutes, about 3 minutes to about 20 minutes, about 3 minutes to about 15 minutes, about 3 minutes to about 10 minutes, or about 5 minutes to about 10 minutes. Those skilled in the art will, by considering various factors such as the starting and desired color of the hair, be able to determine an appropriate amount of time to leave the ready-to-use hair coloring composition on the hair to achieve the desired result. After the ready-to-use hair coloring composition is allowed to remain on the hair for a period of time, the ready-to-use hair coloring composition may, optionally be rinsed or cleansed (e.g., shampooed) from the hair.

### Forms

The hair coloring compositions of the instant disclosure (hair coloring base compositions, developer compositions, and ready-to-use hair coloring compositions) can be in a variety of forms. For example, in many instances, the compositions are in the form of a liquid, gel, lotion, crème, and/or spray. The compositions may be packaged in a variety of different containers. Nonlimiting examples of useful packaging include tubes, jars, caps, unit dose packages, and bottles, including squeezable tubes and bottles.

### Kits

The hair coloring base compositions of the instant disclosure may be present in a kit. For example, in certain embodiments, such kits include: (i) one or more hair coloring base compositions according to the instant disclosure; and (ii) one or more developer composition comprising one or more oxidizing agents; wherein the one or more hair coloring base compositions of (i) and the one or more developer compositions of (ii) are separately contained.

In some embodiments, the developer composition is aqueous or is in the form of an emulsion. The developer composition can contain at least one solvent, chosen from water, organic solvents, and mixtures thereof.

In various exemplary embodiments, hydrogen peroxide is present in an amount of at least about 1% by weight, based on the total weight of the developer composition. In further embodiments, hydrogen peroxide is present in an amount ranging from about 0.1% to about 80% by weight, such as from about 1.0% to about 75% by weight, or from about 2% to about 10% by weight, based on the total weight of the developer composition. In further exemplary embodiments, the hydrogen peroxide may be present in the developer composition in an amount ranging from about 2% to about 25%, such as about 4% to about 20%, about 6% to about 15%, or about 7% to about 10%.

A cosmetically acceptable carrier for the developer composition may, for example, be present in an amount ranging from about 0.5% to about 99% by weight, such as from about 5% to about 95% by weight, relative to the total weight of the developer composition.

In alternative embodiments, the developer composition is substantially anhydrous. The term “substantially anhydrous” means that the developer composition is either completely free of water or contains no appreciable amount of water, for example, no more than 5% by weight, or no more than 2% by weight, or no more than 1% by weight, based on the weight of the developer composition. It should be noted that this refers, for example, to bound water, such as the water of crystallization of the salts or traces of water absorbed by the raw materials used in the preparation of the compositions according to embodiments of the disclosure.

When the developer composition is substantially anhydrous, the developer composition may comprise at least one solvent chosen from organic solvents. Suitable organic solvents for use in the developer composition include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The developer composition may be in the form of a powder, gel, liquid, foam, lotion, cream, mousse, and emulsion.

The pH of the developer composition can range from about 2 to about 12, such as from about 3 to about 7, and it may be adjusted to the desired value using acidifying/alkalizing agents that are well known in the art.

### EXAMPLES

Implementation of the present disclosure is provided by way of the following examples. The examples serve to illustrate the technology without being limiting in nature.

### Example 1

(Hair Coloring Base Compositions)

| | | A | B | C | D | E | F | G | H | I |
|---|---|---|---|---|---|---|---|---|---|---|
| Oxidative Dye | 2-METHOXYMETHYL-P- | 0.8 | 1.9 | 3.2 | 2.7 | 3.1 | 3.1 | 3.1 | 3.1 | 3.0 |
| Precursor | PHENYLENEDIAMINE | | | | | | | | | |
| | 2,3-DIAMINODIHYDRO-PYRAZOLO | | | | | 0.3 | 0.3 | 0.3 | 0.5 | 0.6 |
| | PYRAZOLONE DIMETHOSULFONATE | | | | | | | | | |
| Alkalizing Agent | MONOETHANOLAMINE | 0.935 | 1.06 | 1.02 | 0.66 | qs | qs | qs | 0.7 | 0.7 |
| | | | | | | pH 7.5 | pH 8.7 | pH 9.5 | | |
| Reducing Agent | AMMONIUM THIOLACTATE | 0.5 | 0.5 | 0.5 | 0.5 | 0.5 | 0.5 | 0.5 | 0.5 | 0.5 |
| Fatty Alcohol | OLEYL ALCOHOL | 1.1 | 1.1 | 1.1 | 1.1 | 1.1 | 1.1 | 1.1 | 1.1 | 1.1 |
| Nonionic | PEG-4 RAPESEEDAMIDE, | 24 | 24 | 24 | 24 | 24 | 24 | 24 | 24 | 24 |
| Surfactants | DECETH-3, POLOXAMER 338, | | | | | | | | | |
| | AND/OR GLYCERYL LAURYL ETHER | | | | | | | | | |
| Anionic Surfactant | LAURETH-6 CARBOXYLIC ACID | 4.5 | 4.5 | 4.5 | 4.5 | 4.5 | 4.5 | 4.5 | 4.5 | 4.5 |
| Water Soluble | DIPROPYLENE GLYCOL, HEXYLENE | 20.4 | 20.4 | 20.4 | 20.4 | 20.4 | 20.4 | 20.4 | 20.4 | 20.4 |
| Organic Solvents | GLYCOL, PROPYLENE GLYCOL, | | | | | | | | | |
| | AND/OR ALCOHOL DENAT. | | | | | | | | | |
| Couplers | 2-AMINO-3-HYDROXYPYRIDINE | | 0.03 | 0.2 | | 0.1 | 0.1 | 0.1 | 0.1 | 0.1 |
| | HYDROXYBENZOMORPHOLINE | 0.2 | 0.5 | 1.1 | 1.3 | 1.5 | 1.6 | 1.5 | 1.4 | 1.5 |
| | 2-METHYL-5- | 0.08 | 0.2 | | 0.8 | 0.8 | 0.8 | 0.8 | 1.0 | 0.8 |
| | HYDROXYETHYLAMINOPHENOL | | | | | | | | | |
| | 2-METHYLRESORCINOL | 0.5 | 0.9 | 1.0 | | | | | | |
| | 2,4-DIAMINOPHENOXYETHANOL HCL | | 0.3 | 0.2 | 0.3 | 0.5 | 0.5 | 0.5 | 0.6 | 0.5 |
| Miscellaneous | FRAGRANCE, EDTA, ERYTHORBIC | 1.3 | 1.3 | 1.3 | 1.3 | 1.3 | 1.3 | 1.3 | 1.3 | 1.3 |
| | ACID (antioxidant) | | | | | | | | | |
| Water | WATER | 45.5 | 43.4 | 41.4 | 42.2 | 41.0 | 41.0 | 41.0 | 40.7 | 40.8 |

| | | | | | | | | | |
|---|---|---|---|---|---|---|---|---|---|
| pH | 7.5 | 7.5 | 9 | 9 | 7.5 | 8.7 | 9.5 | 9 | 9 |
| pH of 1:1 Mixture with Developer of Example 2 | 7.5 | 7.5 | 7.5 | 7.5 | 6.5 | 7.5 | 10 | 7.5 | 7.5 |

### Example 2

### Developer Compostion

20 V Developer

| | | | WT. % |
|---|---|---|---|
| (a) | Oxidizing | HYDROGEN PEROXIDE | 7 |
| | Agent | | |
| (c) | Water-Soluble | GLYCERIN | 0.5 |
| | Solvent | | |
| (d) | Fatty Alcohol | CETEARYL ALCOHOL | 2.28 |
| (e) | Nonionic | TRIDECETH-2 CARBOXAMIDE | 2 |
| | Surfactants | MEA | |
| | | CETEARETH-25 | 0.57 |
| (f) | Miscellaneous | SODIUM SALICYLATE | 0.035 |
| | | TETRASODIUM | 0.04 |
| | | PYROPHOSPHATE | |
| | | TETRASODIUM ETIDRONATE | 0.2 |
| (b) | Water | WATER | 83.525 |

### Example 3

### Tenacity Study

The hair coloring base compositions E, F, and G of Example 1 were mixed with the developer composition of Example 2 in a 1:1 ratio to form ready-to-use hair coloring compositions. The hair coloring base compositions E, F, G of Example 1 are identical except for the amount of ethanolamine. The higher amounts of ethanolamine included in hair coloring base compositions F and G resulted in these compositions having a higher pH than the hair coloring base composition E. The three ready-to-use hair coloring compositions are summarized in the table below.

| | Ready-to-Use | | |
|---|---|---|---|
| | Coloring | | |
| | Composition | Content | pH |
| #1 | 1:1 Mixture of Base | Composition E of Example 1 | 7.5 |
| | E and Developer | mixed with Developer of | |
| | | Example 2 in a 1:1 ratio | |
| #2 | 1:1 Mixture of Base | Composition F of Example 1 | 6.3 |
| | F and Developer | mixed with Developer of | |
| | | Example 2 in a 1:1 ratio | |
| #3 | 1:1 Mixture of Base | Composition G of Example 1 | 10 |
| | G and Developer | mixed with Developer of | |
| | | Example 2 in a 1:1 ratio | |

Each of the three hair coloring compositions were mixed with International 20V Developer at a mix ratio of 1:1. This mixture was applied to 90% grey hair swatches, which are commercially available. The mixtures were allowed to remain on the hair of the swatches (to process) for 5 minutes before being rinsed from the hair with water. The hair swatches were subsequently washed with a standard sulfate-base hair cleansing composition. The washing and drying procedure was repeated for 15 cycles, i.e., the hair swatches were washed with the standard sulfate-based hair cleansing composition and subsequently dried 15 consecutive times. Color measurements were taken and images were captured of the swatches before application, after the initial treatment with the ready-to-use hair coloring compositions, as well as after 5, 10, and 15 washes.

The color retention on the hair swatches was assessed as a function of washing using CIE L*a*b* coordinates. EE represents a difference in color, where a greater ΔE value represents increased removal of color or decreased retention of color. ΔE is defined by the following equation:

ΔE=\sqrt{(L*−L₀*)²+(a*−a₀*)²+(b*−b₀*)²}

where L*, a*, and b* represent values measured after a treatment, and L₀*, a₀*, and b₀* represent values measured before treatment(s).

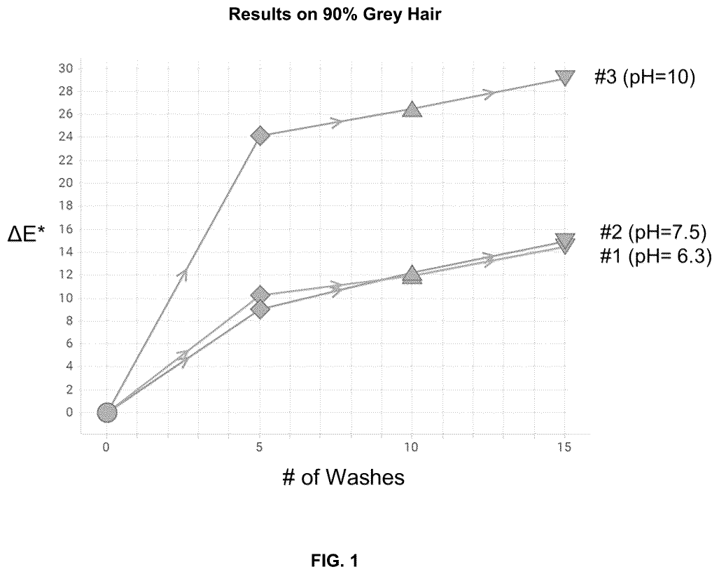

The color of the hair was analyzed before treatments with the ready-to-use hair coloring composition; immediately after the initial treatment with the ready-to-use hair coloring compositions; and after being washed 5, 10, and 15 times. The results are shown in FIG. 1.

As illustrated by the data, the ready-to-use hair coloring compositions #1 and #2, having a pH of 7.5 and 6.3 respectively (prepared using hair coloring base compositions E and F), provided significantly better color retention on grey hair than the ready-to-use hair coloring composition #3 having a higher pH of 10 (prepared using hair coloring base composition G).

### Example 4

### Tenacity Study

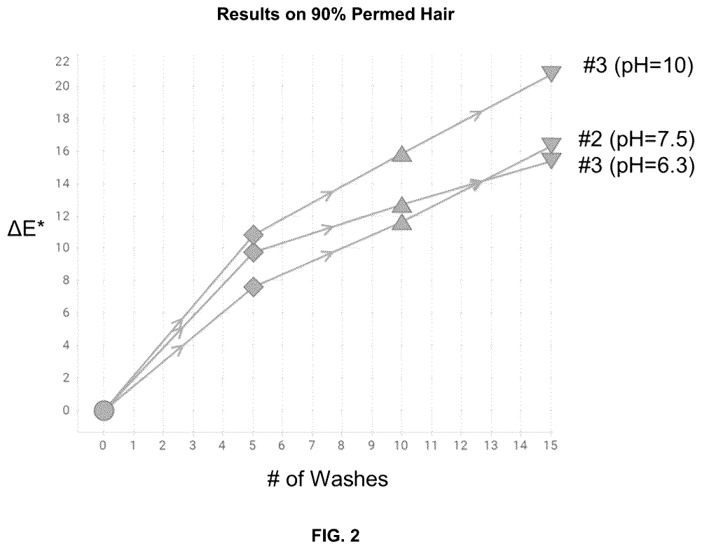

The procedure describe above in Example 3 was repeated except that the three ready-to-use hair coloring compositions were applied to 90% permed hair swatches (which are commercially available) instead of 90% bleached hair. The results are shown in FIG. 2.

As illustrated by the data, the ready-to-use hair coloring compositions #1 and #2, having a pH of 7.5 and 6.3 respectively (prepared using hair coloring base compositions E and F), provided significantly better color retention on permed hair than the ready-to-use hair coloring composition #3 having a higher pH of 10 (prepared using hair coloring base composition G).

### Definitions

The term “hair” as used herein includes hair of the head, beard hair, mustache hair, eyebrow hair, eyelashes, and body hair, unless otherwise specified.

The term “beard hair” includes mustache hair, unless otherwise specified.

As used herein, the terms “comprising,” “having,” and “including” are used in their open, non-limiting sense.

A “hair coloring base composition” as used herein is a hair coloring composition containing one or more oxidative dye precursors and is mixed with a developer composition to form a ready-to-use hair coloring composition.

A “developer composition” as used herein is a composition containing one or more oxidizing agents, preferably a peroxide (hydrogen peroxide) and is missed with a hair coloring base composition to form a ready-to-use hair coloring composition.

A “ready-to-use hair coloring composition” is an “active” composition that includes one or more oxidative dye precursors and one or more oxidizing agents; and is formed by combining a hair coloring base composition with a developer composition.

A “composition colorant” is a compound that colors the composition but does not have an appreciable coloring effect on hair. In other words, the composition colorant is included to provide color to the composition, for example, for aesthetic appeal. It is not included to impart color to the hair. Styling gels, for example, can be found in a variety of different colors (e.g., light blue, light pink, etc.) yet application of the styling gel to the hair does not change the color of the hair.

“Oil” is used herein to refer to an organic compound insoluble in water at normal temperature (25° C.) and at atmospheric pressure (760 mmHg), i.e. it has a water solubility of less than 5% by weight, or less than 1% by weight, or less than 0.1% by weight. Oils have in their structure a chain of at least two siloxane groups or at least one hydrocarbon chain having at least 6 carbon atoms. Furthermore, oils are generally soluble in organic solvents in the same conditions of temperature and pressure, for example in chloroform, ethanol, benzene or decamethylcyclopentasiloxane. Furthermore, oils are liquid at ordinary temperature (25° C.) and at atmospheric pressure (760 mmHg). The oils preferably do not contain any carboxylic acid functions, i.e. they do not contain any —COON or —COO— groups. As described throughout the disclosure fatty alcohols are independent from fatty compounds and oils, i.e., even if a fatty alcohol is present in the compositions of the instant disclosure, the compositions may nonetheless be free or essentially free from fatty compounds of oils (because fatty alcohols are not included in the definition of fatty compounds and oils).

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

Some of the various categories of components identified may overlap. In such cases where overlap may exist and the composition includes both components (or the composition includes more than two components that overlap), an overlapping compound does not represent more than one component. For example, a fatty acid may be considered both an emulsifier and a fatty compound. If a particular composition includes both an emulsifier and a fatty compound, a single fatty acid will serve as only the emulsifier or only the fatty compound (the single fatty acid does not serve as both the emulsifier and the fatty component).

The salts referred to throughout the disclosure may include salts having a counter-ion such as an alkali metal, alkaline earth metal, or ammonium counter-ion. This list of counter-ions, however, is non-limiting.

The term “treat” (and its grammatical variations) as used herein refers to the application of the compositions of the present disclosure onto the surface of keratinous substrates such as hair. The term ‘treat” (and its grammatical variations) as used herein also refers to contacting keratinous substrates such as hair with the compositions of the present disclosure.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

All components positively set forth throughout the instant disclosure may be negatively excluded from the claims, e.g., a claimed composition may be “free,” “essentially free” (or “substantially free”) of one or more components that are positively set forth in the instant disclosure. As an example, silicones can optionally be included in the compositions but preferably the compositions are free or essentially free from silicones. Silicones are synthetic polymers made up of repeating units of siloxane, elemental silicon and oxygen, combined with other elements, most often carbon and hydrogen. Thus, silicones are also called polysiloxanes.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickeners and/or ethoxylated surfactants. Likewise, a particular composition may be free or essentially free of sulfates, such as sulfate surfactants.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair coloring base composition comprising:
(a) 2-methoxymethyl-P-phenylenediamine, and optionally one or more additional oxidative dye precursors;
(b) one or more couplers; and
(c) 3 wt. % or less of one or more alkalizing agents;
(d) about 0.1 to about 10 wt. % of one or more fatty alcohols having from 12 to 24 carbon atoms;
(e) about 5 to about 40 wt. % of one or more surfactants;
(f) about 10 to about 40 wt. % of one or more water-soluble organic solvents;
(g) about 25 to about 60 wt. % water;
wherein (e), (f), and (g) are in amounts such that a combined amount of (e), (f), and (g) constitutes at least 70 wt. % of the hair coloring base composition;
the hair coloring base composition forms a ready-to-use hair coloring composition having a pH from about 6 to about 8 upon mixing with a developer composition comprising hydrogen peroxide in a weight ratio of about 1:5 to about 5:1 (hair coloring base composition:developer composition); and
all percentages by weight are based on the total weight of the hair coloring base composition.

2. The hair coloring base composition of claim 1, wherein the alkalizing agent is an organic alkalizing agent.

3. The hair coloring base composition of claim 1 having a pH from about 7 to about 10.

4. The hair coloring base composition of claim 1, wherein the one or more oxidative dye precursors of (a) and the one or more couplers of (b) are in a weight ratio of about 1:1 to about 5:1 ((a):(h)).

5. The hair coloring base composition of claim 1, wherein the one or more alkalizing agents are chosen from ammonia, ammonium compounds, percarbonate salts, alkanolamines, alkali metal carbonates, sodium silicate, sodium metasilicate, and a mixture thereof.

6. The hair coloring base composition of claim 1, wherein the one or more alkalizing agents are chosen from alkanolamines.

7. The hair coloring base composition of claim 6, wherein the alkanolamines are chosen from monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol, and a mixture thereof.

8. The hair coloring base composition of claim 1 being free from ammonia and ammonium ions.

9. The hair coloring base composition of claim 1, wherein the one or more fatty alcohols are chosen from cetyl alcohol, stearyl alcohol, cetearyl alcohol, behenyl alcohol, lauryl alcohol, myristic or myristyl alcohol, arachidyl alcohol, lignoceryl alcohol, oleyl alcohol, and mixtures thereof.

10. The hair coloring composition of claim 1 comprising:
(e)(1) about 15 to 30 wt. % of one or more nonionic surfactants; and
(e)(2) about 1 to about 10 wt. % one or more anionic surfactants.

11. The hair coloring composition of claim 1, wherein the one or more water-soluble organic solvents are chosen from glycerin, mono-alcohols, polyols (polyhydric alcohols), glycols, and a mixture thereof, preferably, the one or more water-soluble solvents are chosen from glycerin, propylene glycol, butylene glycol, pentylene glycol, dipropylene glycol, hexylene glycol, ethanol, isopropanol, t-butyl alcohol, and a mixture thereof.

12. The hair coloring composition of claim 1, wherein the one or more couplers are chosen from aromatic meta-diamines, meta-aminophenols, meta-diphenols, heterocyclic compounds, indole compounds, and mixtures thereof.

13. The hair coloring base composition of claim 1 being free from resorcinol and derivatives thereof.

14. The hair coloring base composition of claim 1, further comprising:
(h) one or more reducing agents.

15. The hair coloring base composition of claim 16, wherein the one or more reducing agents are chosen from potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof.

16. A kit comprising:
(i) one or more hair coloring base compositions of claim 1; and
(ii) one or more developer compositions comprising:
(a) one or more oxidizing agents; and
(b) water;
wherein the one or more hair coloring base compositions of (i) and the one or more developer compositions of (ii) are separately contained.

17. A ready-to-use hair coloring composition comprising a mixture of:
(i) the hair coloring base composition of claim 1; and
(ii) a developer composition comprising:
(a) one or more oxidizing agents; and
(b) water;
wherein the ready-to-use hair coloring composition has a pH of about 6 to about 8, preferably about 6.2 to about 7.8, more preferably about 6.5 to about 7.5.

18. A method for making a ready-to-use hair coloring composition comprising mixing a hair coloring base composition of claim 1 with a developer composition in a weight ratio of about 1:5 to about 5:1 (hair coloring base composition:developer composition) to form a ready-to-use hair coloring composition having a pH of about 6 to about 8, wherein the developer composition comprises:
(a) one or more oxidizing agents; and
(b) water.

19. A method for coloring hair comprising:
(i) obtaining the hair coloring base composition of claim 1;
(ii) obtaining a developer composition comprising:
(a) one or more oxidizing agents; and
(b) water;
(iii) mixing the hair coloring base composition of (i) and the developer composition of (ii) in a weight ratio of about 1:5 to about 5:1 to form a ready-to-use hair coloring composition having a pH of about 6 to about 8; and
(iv) applying the ready-to-use hair coloring composition to hair;
(v) allowing the ready-to-use hair coloring composition to remain on the hair for a period of time, wherein the period of time is less than 30 minutes, preferably less than 15 minutes, more preferably less than 10 minutes; and
(vi) after the period of time has lapsed, rinsing the ready-to-use hair coloring composition from the hair.

20. The method of claim 19, wherein the hair is beard or mustache hair.
